(19) United States
(12) Patent Application Publication
(10) Pub. No.: US 2011/0091910 A1
(43) Pub. Date: Apr. 21, 2011

(54) NOVEL ASSAY

(75) Inventors: Martin Kleinschmidt, Halle / Saale (DE); Claudia Goettlich, Halle / Saale (DE); Hans-Ulrich Demuth, Halle / Saale (DE)

(73) Assignee: PROBIODRUG AG, Halle / Saale (DE)

(21) Appl. No.: 12/884,403

(22) Filed: Sep. 17, 2010

Related U.S. Application Data
(60) Provisional application No. 61/243,604, filed on Sep. 18, 2009.

(51) Int. Cl. G01N 33/566

(52) U.S. Cl. 435/7.92; 436/501

(57) ABSTRACT

A novel method for detection of Aβ peptides, in particular in plasma, and to the use of Aβ peptides for diagnosis of Alzheimer's disease.

### CROSS-REFERENCE TO RELATED APPLICATIONS

This application claims priority from U.S. Provisional Application Ser. No. 61/243,604, filed on Sep. 18, 2009, which is incorporated herein by reference in its entirety.

### MATERIAL INCORPORATED BY REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally concerns the detection and diagnosis of Alzheimer's disease with the use of Aβ(1-40) as a biomarker and further concerns a novel method to determine Aβ(1-40) in biological samples.

### BACKGROUND OF THE INVENTION

Alzheimer's disease is the most common form of dementia and has a prevalence of approximately 65-70% among all dementia disorders (Blennow et al., 2006). Resulting from increased life expectancy, this disease has become a particular issue in highly developed industrialised countries like Japan and China as well as in the US and Europe. The number of Alzheimer patients is estimated to increase from 24 million in 2001 to 81 million in 2040 (Ferri et al., 2005). Currently, the costs for treatment and care of AD patients worldwide amount to approximately 250 billion US dollars per year.

The progression of the disease is relatively slow and Alzheimer's disease will usually last for about 10-12 years after the onset of first symptoms. Presently, it is extremely difficult to make a reliable and early diagnosis of AD and distinguish it from other forms of dementia. A good diagnosis with a reliability of more than 90% is only possible in the later stages of the disease. Prior to that, it is only possible to make a prediction that Alzheimer's is possible or probable; diagnosis here relies on the use of certain criteria according to Knopman et al., 2001; Waldemar et al., 2007 or Dubois et al., 2007. Neurodegeneration starts however 20 to 30 years before the first clinical symptoms are noticed (Blennow et al., 2006; Jellinger K A, 2007). The onset of the clinical phase is usually characterised by the so-called “mild cognitive impairment” (MCI), where patients will show measurable cognitive deficits which are not sufficient to enable a diagnosis of a dementia disease in a clear fashion (Petersen et al., 1999; Chetkow et al., 2008). Many patients with MCI will have neuropathological changes which are typical for AD and which means that an earlier stage of AD is possible, but not certain (Scheff et al., 2006; Markesbery et al., 2006; Bouwman et al., 2007). There are however many MCI cases which will not progress to Alzheimer's; in these cases, other factors are responsible for the cognitive deficit (Saito et al., 2007; Jicha et al., 2006 and Petersen et al., 2006). While some MCI patients will not show any deterioration of their condition or even some kind of amelioration, for most MCI cases the cognitive deficit will continue to clinical dementia. The yearly rate of this conversion is approximately 10-19% (Gauthier et al., 2006; Fischer et al., 2007). At present there is a combination of clinical, neuropsychological and imaging processes which are capable of differentiating the various subtypes of Mild Cognitive Impairment (Devanand et al., 2007; Rossi et al., 2007; Whitwell et al., 2007; Panza et al., 2007). However, there is no significant difference between these subtypes in relation to the further progression of dementia (Fischer et al., 2007). Thus, it is of utmost importance to develop a method to enable a clear and reliable diagnosis of Alzheimer's disease in the early stages, preferably at its onset or during MCI.

Prior Art Biomarkers

Biomarkers for Alzheimer's disease have already been described in the prior art. Alongside the well known psychological tests like e.g. ADAS-cog, MMSE, DemTect, SKT or the Clock Drawing test, biomarkers are supposed to improve diagnostic sensitivity and specificity for first diagnosis as well as for supervising the progression of the disease. In relation to the current status of development of biomarkers for AD/MCI it was proposed to correlate the disease in the future with the other diagnostic criteria (Whitwell et al., 2007; Panza et al., 2007; Hyman S E, 2007). Biomarkers are supposed to support or to replace the classical neuro-psychological tests in the future. There is a common belief that they will be of great importance as surrogate markers for the development of agents against Alzheimer's (Blennow K, 2004; Blennow K, 2005; Hampel et al., 2006; Lewczuk et al., 2006; Irizarry M C, 2004).

Structural Biomarkers

The “Magnetic resonance imaging” (MRI) is an imaging process which allows detection of degenerative atrophies in the brain (Barnes J et al., 2007; Vemuri et al., 2008). Thus, atrophy of the medial temporal lobe (MTA) is sensitive to a degeneration of the hippocampal region in the brain of older patients; this can be made visible very clearly by MRI, but is not specific for Alzheimer's disease. Mild MTA is not encountered more frequently in other dementias (Barkhof et al., 2007) but it does correlate with MCI (Mevel et al., 2007). For this reason, it is not possible to determine from MRI data alone, whether the neurodegeneration is Alzheimer's disease or an early stage of Alzheimer's disease. A further imaging method is the Positron Emission Tomography (PET), which allows making the accumulation of a detector molecule (PIB) on amyloid deposits visible. It has been detected that the thioflavine T-analogue (¹³C)PIB is accumulated in certain regions of the brain of patients with MCI or mild Alzheimer's disease, respectively (Kemppainen et al., 2007; Klunk et al., 2004; Rowe et al., 2007). However, this is also detectable in subjects, who do not show symptoms of dementia (Pike et al., 2007). This, in turn, would probably indicate that the detection of amyloid deposits via PET allows the detection of pre-clinical stages of Alzheimer's disease; if this phenomenon will be confirmed in further studies. Besides the most frequently used processes, MRI and PET, there are additional structural biomarkers for Alzheimer's disease known: CBF-SPECT, CMRg 1-PET (glucose metabolism proton spectroscopy (H-1 MRS), high field strength functional MRI, voxel-based morphometry, enhanced activation of the mediobasal temporal lobe (detected by fMRI, (R)-[(¹¹)C]PK11195 PET for the detection of microglial cells (Huang et al., 2007; Kantarci et al., 2007; Petrella et al., 2007; Hamalainen et al., 2007; Kircher et al., 2007; Kropholler et al., 2007).

CSF Biomarkers

Senile plaques are one of the pathological characteristics of Alzheimer's disease. These plaques consist mostly of Aβ(1-42) peptides (Attems J, 2005). In some studies it could be shown that a low level of Aβ(1-42) in CSF of MCI patients correlates specifically with the progression of Alzheimer's disease (Blennow and Hampel, 2003; Hansson et al., 2006 and 2007). The reduction in CSF is probably due to enhanced aggregation of Aβ(1-42) in the brain (Fagan et al., 2006; Prince et al., 2004; Strozyk et al., 2003). Another possible explanation is the occurrence of semi-soluble Aβ(1-42) oligomers (Walsh et al., 2005), which would lead to a lower level of detection in CSF. Particularly in the early stages of Alzheimer's, decreased concentrations of Aβ(1-42) would be detected, while increased amounts of Tau protein and phospho-Tau proteins in CSF, respectively, could be detected (Ewers et al., 2007; Lewczuk et al., 2004). To provide a better predictability of biomarkers, it is usually attempted to use the Tau/Aβ(1-42) ratio and correlate it with the prediction of cognitive deficiency in older persons, who do not have dementia (Fagan et al., 2007; Gustafson et al., 2007; Hansson et al., 2007; Li et al., 2007; Stomrud et al., 2007), as well as in MCI patients (Hampel et al., 2004; Maccioni et al., 2006; Schönknecht et al., 2007). A further correlation between ante mortem CSF levels of Aβ(1-42), Tau, phospho-Tau-Thr231 and post-mortem histopathological alterations of the brain were detected in AD patients (Clark et al., 2003; Buerger et al., 2006). In other studies, however, no correlation between CSF biomarkers and Aβ(1-42), total Tau and phospho-Tau with APOE ε4-allele, plaque and tangle load after autopsy could be detected (Engelborghs et al., 2007; Buerger et al., 2007). An interesting aspect was detected in a multicenter study. It appears that increased level of total Tau and phospho-Tau (181) correlates with a decreased ratio of Aβ(1-42)/Aβ(1-40), but not with the Aβ(1-42) level alone (Wiltfang et al., 2007). An increased level of CSF Tau was however also detected in other CNS diseases like Creutzfeldt-Jakob disease, brain infarction, and cerebral vascular dementia, which are all associated with a neuronal loss (Buerger et al., 2006 (2); Bibl et al., 2008). A further possible biomarker is the increase of BACE 1 activity in CSF as an indicator for MCI (Zhong et al., 2007). It is also discussed that the increased BACE 1 activity will result in increased Aβ production and therefore increased aggregation of the peptides. Alzheimer's disease is supposed to be accompanied by neuroinflammatory processes. Anti-microglial cell antibodies in the CSF are therefore possible biomarkers for these inflammatory processes in AD (McRea et al., 2007).

In spite of the multitude of biomarkers, which are supposed to enable early diagnosis of Alzheimer's disease, there is currently no single biomarker available that ensures a reliable and clear diagnosis of this disease. Therefore, most studies in the field of Alzheimer's disease use a comparison of the results of the determination of the respective biomarkers and clinical diagnosis to determine the stage and severity of Alzheimer's disease. In contrast, be the correlation of biomarkers with the pathological causes of Alzheimer's disease would deliver a clearly more reliable diagnosis of the disease.

Such a possible approach could be the repeated analysis of immune-precipitated CSF samples of clearly identified and defined neuropathological dementia diseases to clarify, whether Aβ(1-40) and Aβ(1-42) are in fact suitable neurochemical dementia markers (Jellinger et al., 2008). In order to discover novel, up to now unknown, biomarkers for Alzheimer's disease, CSF samples are usually analysed via a comparative proteomic analysis to result in a diagnosis of AD with enhanced sensitivity and also to enable the differentiation from other degenerative dementia disorders (Finehout et al., 2007; Castano et al., 2006; Zhang et al., 2005; Simonsen et al., 2007; Lescuyer et al., 2004; Abdi et al., 2006). After a proteomic analysis the potential new biomarker have to be analysed in detail for its suitability and correlation with pathological causes. A typical example for a biomarker, which was found by a proteomic analysis, is truncated cystatin C as a biomarker for multiple sclerosis. This biomarker was later proven to be a storage artefact (Irani et al., 2006; Hansson et al., 2007(2)).

Plasma Biomarkers

Besides the frequently used plasma biomarkers, i.e. the Aβ peptides, further inflammatory plasma markers are used for the early diagnosis of dementia (Ravaglia et al., 2007; Engelhart et al., 2004), and in particular for Alzheimer's disease (Motta et al., 2007). The suitability and specificity of these inflammatory markers for the diagnosis of Alzheimer's disease is still in discussion. Further possible biomarkers were also found via comparative proteomic analysis of plasma from AD patients and healthy controls (German et al., 2007; Ray et al., 2007). No convincing or suitable data for any of the aforementioned biomarkers are available so far. Contrary to the analysis of amyloid β in CSF, the results achieved up to now with respect to suitable Aβ biomarkers in plasma are not reliable or clear. In some studies a correlation between a decreased ratio of Aβ(1-42)/Aβ(1-40) in plasma and an enhanced conversion of cognitive normal persons to MCI or Alzheimer patients, respectively, was found (Graff-Radford et al., 2007; van Oijen et al., 2006; Sundelof et al., 2008). Other studies however detected that a reduction of the Aβ(1-42) plasma level is more likely a marker for the conversion from MCI to AD (Song et al., 2007) and is not suitable as a marker for neurodegenerative purposes, which are encountered with Alzheimer's disease (Pesaresi et al., 2006). Most of the studies however do not show a difference in Aβ plasma levels between healthy controls and patients with sporadic Alzheimer's disease (Fukumoto et al., 2003; Kosaka et al., 1997; Scheuner et al., 1996; Sobow et al., 2005; Tamaoka et al., 1996; Vanderstichele et al., 2000). Some studies also showed that the level of AP in plasma does not correlate with the level as encountered in the brain (Fagan et al., 2006; Freeman et al., 2007) nor does it correlate with the level encountered in CSF (Mehta et al., 2001; Vanderstichele et al., 2000). In a recent study, a correlation was detected for Aβ(1-40) and Aβ(1-42) between CSF and plasma, but only in healthy controls. This correlation could not be detected in MCI and AD which is explained by destroying the balance between CSF and plasma Aβ due to Aβ deposits in the brain (Giedraitis et al., 2007). Generally, it is assumed that plasma Aβ(1-42) level is not a reliable biomarker for MCI or AD (Blasko et al., 2008; Mehta et al., 2000; Brettschneider et al., 2005), whereas a decrease of the ratio plasma Aβ(1-38)/Aβ(1-40) is considered a biomarker for vascular dementia and comes close to the predictability of CSF markers (Bibl et al., 2007).

EP2020602 and US20020182660 disclose methods to detect specific full-length Aβ peptides, such as Aβ(1-40) and Aβ(1-42), in plasma samples. The methods employ two different capture antibodies, but do not comprise an immuneprezipitation step.

EP1944314 discloses methods to detect autoantibodies against Aβ peptides, such as autoantibodies against Aβ(21-37) or Aβ(4-10). In said methods, polypeptides comprising an amino acid sequence of an Aβ peptide are immobilized on a carrier in order bind and detect the respective autoantibodies.

Hence, there is a clear need for a biomarker, which is reliable and leads to a clear predictability of the early onset of Alzheimer's disease stages as well as differentiation of Alzheimer's disease from other dementia diseases. There is also a need to provide said biomarker from plasma, which is easily obtainable from a patient in contrast to CSF. In addition, there is a need to provide a method for the detection of said biomarker, which leads to a reliable and clear determination of said biomarker.

However, such a method, especially with plasma Aβ as a biomarker, is extremely difficult to establish, because the Aβ peptides are very hydrophobic. All currently known and described assay systems and methods achieve only a very unsatisfactory analytical sensitivity and encounter great problems with the very complex interactions between analytes and matrix, i.e. plasma. This explains the very contradictory results in the multiple studies described above. It has further led to the belief in the scientific community that the level of specific Aβ peptides in plasma is not suitable as a biomarker for AD.

ELISA or ELISA-type systems (Muliplex) are used conventionally for the quantification of Aβ in plasma. The validation parameters of such studies are usually only unsatisfactorily analysed or are completely disregarded. For example, a critical item like the recovery rate is not analysed or is not mentioned in respective publications. The recovery rate is however a decisive parameter for the correct determination of the level of those Aβ peptides, which occur in plasma. Differences in the levels of Aβ peptides in plasma, which occur in different studies, may thus result from the incorrect determination of the recovery rates. A further important parameter of an ELISA or multiplex system is its linearity. For example, in Hansson et al., 2008, the level of the calculated plasma Aβ(1-42) concentration for a 1:20 dilution was three times higher than in the 1:2 dilution of the same sample (Hansson et al., 2008). This example alone shows that different dilutions of the same plasma samples in several studies known so far are not comparable. Moreover, such methods are completely unsuitable for establishing a reliable diagnostic assay. The use of different dilutions within one study would lead to artefacts and in the end to completely false results. As long as there are no standardized protocol and method for the analysis of plasma Aβ, such studies will also be contradictory and unsuitable for diagnostic methods in the future.

Thus, it is an object of the present invention to provide a novel method, which allows the detection of Aβ, in particular in plasma, with a high reliability.

Moreover, the present invention provides diagnostic markers, which can be determined with reliable methods and can be used for reliable and clear prediction of Alzheimer's disease.

The present invention further provides reliable methods, which are particularly suitable for use in multi-patient studies, wherein biological samples are analyzed in different measurement cycles.

### SUMMARY OF THE INVENTION

The object of the present invention is solved by providing a method for the detection of amyloid β peptide (Abeta or Aβ) in biological samples. The method is characterized in that a biological sample is contacted with at least two different capture antibodies in an immune-precipitation step. The resulting complex is isolated, destructed and, subsequently the captured Aβ peptides are analysed in an Aβ specific ELISA. Preferably, this Aβ specific ELISA is a sandwich-ELISA.

Aβ peptides are liberated from the amyloid precursor protein (APP) after a sequential cleavage by the enzymes β-and γ-secretase. The γ-secretase cleavage results in the generation of the above-mentioned Aβ(1-40) and Aβ(1-42) peptides, which differ in their C-termini and exhibit different potencies of aggregation, fibril formation and neurotoxicity.

The present invention thus provides a method for the determination of the levels of the Aβ(1-40) and Aβ(1-42) peptides. It is likewise envisaged that functional equivalents of Aβ(1-40) or Aβ(1-42) are detected The method of the present invention is particularly suitable for the determination of the level of the Aβ(1-40) and/or functional equivalents thereof.

Thus, according to a preferred embodiment of the above-described method, the Aβ peptide to be determined is selected from the group consisting of Aβ(1-40) (SEQ ID No:1), Aβ(1-42) (SEQ ID No: 2) and functional equivalents thereof.

In a particularly preferred embodiment, the Aβ peptide to be detected is Aβ(1-40) (SEQ ID No: 2).

According to a further preferred embodiment, the biological sample is selected from the group consisting of blood, serum, urine, cerebrospinal fluid (CSF), plasma, lymph, saliva, sweat, pleural fluid, synovial fluid, tear fluid, bile and pancreas secretion. In a particularly preferred embodiment, the biological sample is plasma.

The biological sample can be obtained from a patient in a manner well-known to a person skilled in the art. In particular, a blood sample can be obtained from a subject and the blood sample can be separated into serum and plasma by conventional methods. The subject, from which the biological sample is obtained is suspected of being afflicted with Alzheimer's disease, at risk of developing Alzheimer's disease and/or being at risk of or having any other kind of dementia.

In particular, it is a subject suspected of having Mild Cognitive Impairment (MCI) and/or being in the early stages of Alzheimer's disease.

The present method has several advantages over the methods known in the art, i.e. the method of the present invention can be used to detect Alzheimer's disease at an early stage and to differentiate between Alzheimer's disease and other types of dementia in early stages of disease development and progression. One possible early stage is Mild Cognitive Impairment (MCI). It is impossible with the methods currently known in the art to make a clear and reliable diagnosis of early stages of Alzheimer's disease and, in particular, it is impossible to differentiate between the onset of Alzheimer's disease and other forms of dementia in said early stages. This especially applies for patients afflicted with MCI.

In contrast, the methods provided by the present invention are suitable for a differential diagnosis of Alzheimer's disease. In particular, the present invention provides a method, wherein Aβ peptides can be detected in biological samples obtained from any of the above described subjects in a highly reproducible manner. The high reproducibility of the methods of the present invention is achieved by using at least two different capture antibodies in an initial immune-precipitation step. Preferably, these at least two different capture antibodies are directed to different epitopes of the Aβ target peptide.

It is particularly preferred to use Aβ(1-40) in the methods of the present invention.

In a further preferred embodiment, the biological sample is plasma.

The above-mentioned “Aβ target peptide” encompasses Aβ(1-40) and Aβ(1-42) including all functional equivalents thereof.

A specific problem, which had to be overcome by the present invention, is that the biomarker to be used is altered in early stages of Alzheimer's disease, e.g. during mild cognitive impairment. The inventors of present invention have shown that it is possible to determine Aβ peptides, in particular Aβ(1-40), in a reliable manner, and, it also became clear for the first time that in fact Aβ(1-40) is particularly suitable for the diagnosis of early onset Alzheimer's disease.

Moreover, it became clear with the present invention that the level of Aβ(1-40) is an initial and early marker for the onset of early stage Alzheimer's disease, because it's plasma level is increased during the early stages of Alzheimer's disease and is especially high in persons categorized with mild cognitive impairment. Only with the present invention is it possible to show that high plasma concentrations of Aβ(1-40) were associated with a positive clinical diagnosis of Alzheimer's disease. This is contrary to the earlier belief in the prior art that Aβ(1-40) is not a suitable marker for AD as the attempts to show a correlation ended with statistically insignificant data and without establishing any statistically significant correlation.

These surprising results of the present invention were achieved by using the present inventive method, which employs the novel bivalent capture system for the initial immuneprecipitation step. This bivalent capture system is defined by two antibody molecules, or more than two antibody molecules, recognising at least two different epitopes of the Aβ peptides. Thus, it has been shown that a significant increase of Aβ(1-40) is an early event in the progression of Alzheimer's disease. According to a preferred embodiment, the at least two different capture antibodies are each specific for a different epitope of the Aβ peptide, in particular the Aβ(1-40) peptide.

The immuneprecipitation step is advantageous for several reasons: It reduces matrix effects, i.e. it eliminates impurities, which are normally comprised in each biological sample, thereby making the methods of the present invention more sensitive. Further, the immuneprecipitation step leads to the preconcentration of the Aβ peptides, which results in an increased affinity of the subsequently used detection antibodies.

Other objects and features will be in part apparent and in part pointed out hereinafter.

### DEFINITIONS

“Capture antibody” in the sense of the present application is intended to encompass those antibodies which bind to a target Aβ peptide.

Preferably the capture antibodies bind to the Aβ peptide with a high affinity. In the context of the present invention, high affinity means an affinity with a K_{D} value of 10⁻⁷M or better, preferably a K_{D} value of 10⁻⁸M or better or even more preferably, a K_{D} value of 10⁻⁹M to 10⁻¹²M.

The term “antibody” is used in the broadest sense and specifically covers intact monoclonal antibodies, polyclonal antibodies, multispecific antibodies (e.g. bispecific antibodies) formed from at least two intact antibodies, and antibody fragments as long as they exhibit the desired biological activity. The antibody may be an IgM, IgG (e.g. IgG1, IgG2, IgG3 or IgG4), IgD, IgA or IgE, for example. Preferably however, the antibody is not an IgM antibody. The “desired biological activity” is binding to an Aβ peptide.

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single determinant on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Köhler et al., Nature, 256:495 (1975), or may be made by generally well known recombinant DNA methods. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain a minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences.

These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986), Reichmann et al, Nature. 332:323-329 (1988): and Presta, Curr. Op. Struct. Biel., 2:593-596 (1992). The humanized antibody includes a Primatized™ antibody wherein the antigen-binding region of the antibody is derived from an antibody produced by immunizing macaque monkeys with the antigen of interest or a “camelized” antibody.

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{D}) in the same polypeptide chain (V_{H}−V_{D}). By using a linker that is too short to allow pairing between the two domains on the same chain, the domains are forced to pair with the complementary domains of another chain and create two antigen-binding sites. Diabodies are described more fully in Hollinger et al., Proc. Natl. Acad. Sol. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the expressions “cell”, “cell line,” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

“Amyloid β, Aβ or /β-amyloid” is an art recognized term and refers to amyloid β proteins and peptides, amyloid β precursor protein (APP), as well as modifications, fragments and any functional equivalents thereof. In particular, by amyloid β as used herein is meant any fragment produced by proteolytic cleavage of APP but especially those fragments which are involved in or associated with the amyloid pathologies including, but not limited to, Aβ1-38, Aβ1-40, Aβ1-42.

“Functional equivalents” encompass all those mutants or variants of Aβ(1-40) and /or Aβ(1-42) which might naturally occur in the patient group which has been selected to undergo the method for detection or method for diagnosis as described according to the present invention.

More particularly, “functional equivalent” in the present context means that the functional equivalent of Aβ(1-40) or Aβ(1-42) are mutants or variants thereof and have been shown to accumulate in Alzheimer's disease. The functional equivalents have no more than 30, preferably 20, more preferably 10, particularly preferably 5 and most preferred 2, or only 1 mutation(s) compared to Aβ(1-40) and/or Aβ(1-42). Functional equivalents also encompass mutated variants, which comprise by way of example all Aβ peptides starting with amino acids Asp-Ala-Glu and ending with Gly-Val-Val and Val-Ile Ala, respectively.

Particularly useful Aβ(1-40) and Aβ(1-42) equivalents in the present context are those described by Irie et al., 2005, namely the Tottori, Flemish, Dutch, Italian, Arctic and Iowa mutations of Aβ. Functional equivalents also encompass Aβ peptides derived from amyloid precursor protein bearing mutations next to the β- or γ-secretase cleavage site like the Swedish, Austrian, French, German, Florida, London, Indiana and Australian variations (Irie et al., 2005).

“Sandwich ELISAs” usually involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assay). For example, one preferable type of sandwich assay is an ELISA assay, in which case the detectable moiety is an enzyme.

### BRIEF DESCRIPTION OF THE FIGURES

FIG. 1:

Bivalent Immuneprecipitation System improves capture efficiency
(A) Recovery of Aβ1-40 from Cyp18 solution and human Plasma by usage of different antibody combinations
(B) Schematic of bivalent capture system (shaded: 4G8 antibody, grey: x-40 antibody, black: anti-mouse antibody conjugated to magnetic bead).

FIG. 2:

DemTect Test, Mean values (Mean±SD) of the results of classification differences in AD patients and healthy subjects by DemTect Scale.

FIG. 3:

Mini-Mental-State Test, Mean values (Mean±SD) of the results of classification differences in AD patients and healthy subjects by Mini-Mental-State Test.

FIG. 4:

Clock-Drawing Test, Mean values (Mean±SD) of the results of classification differences in AD patients and healthy subjects by Clock-Drawing Test.

FIG. 5:

Plot of Aβ(1-40) concentration vs. the score of DemTect (A) and MMSE (B), respectively.

FIG. 6:

Relative plasma Aβ(1-40) level, normalized to internal plasma standard (ITS), mean values and SEM.

### SEQUENCES

Aβ 1-42 (SEQ ID NO. 1):
Asp-Ala-Glu-Phe-Arg-His-Asp-Ser-Gly-Tyr-Glu-
Val-His-His-Gln-Lys-Leu-Val-Phe-Phe-Ala-Glu-
Asp-Val-Gly-Ser-Asn-Lys-Gly-Ala-Ile-Ile-Gly-
Leu-Met-Val-Gly-Gly-Val-Val-Ile-Ala
Aβ 1-40 (SEQ ID NO. 2):
Asp-Ala-Glu-Phe-Arg-His-Asp-Ser-Gly-Tyr-Glu-
Val-His-His-Gln-Lys-Leu-Val-Phe-Phe-Ala-Glu-
Asp-Val-Gly-Ser-Asn-Lys-Gly-Ala-Ile-Ile-Gly-
Leu-Met-Val-Gly-Gly-Val-Val

### DETAILED DESCRIPTION OF THE INVENTION

The present invention provides a method for the detection and determination of Aβ peptides in biological samples. The method is characterized in that a biological sample is contacted with at least two different capture antibodies in an immuneprecipitation step. The resulting complex is isolated, destructed and, subsequently the captured Aβ peptides are analysed in an Aβ specific ELISA. Preferably, this Aβ specific ELISA is a sandwich-ELISA.

As mentioned above, at least two antibodies for the initial capture step should be selected which have different specificities for different epitopes of the Aβ peptide.

The method of the present invention comprises the following steps:
i) Contacting a biological sample with at least two different capture antibodies in an immuneprecipitation step.
After contacting the biological sample with the aforementioned at least two different capture antibodies, an immune complex will form between the at least two different capture antibodies and the Aβ peptides. This step does not act for specific isolation of full length Aβ(1-40) and/or Aβ(1-42), rather than capturing and separating all Aβ species, especially ending at position 40 and/or position 42.
ii) This complex is then detected by secondary antibodies. Suitably, the secondary antibodies are immobilized on magnetic beads. Together with the magnetic beads the immune complex can be easily separated from the body fluid (plasma/serum CSF etc.) using the magnetic separator.
iii) The immune complex is eluted from the beads. Suitably, the elution step is performed by incubating the beads carrying the immune complex in a solution comprising 50% Methanol/0.5% formic acid for 1 h at room temperature. Thereby, all intermolecular interactions are destructed and all Aβ peptide molecules, which were isolated from the biological sample, are released from the beads in the solution.
iv) The released, isolated Aβ peptide will be quantified in a subsequent step, for example by a sandwich ELISA that specifically detects full length Aβ(1-40) and/or Aβ(1-42).

In multi-patient diagnostics studies, e.g. where more than 100 biological samples have to be analyzed, there have to be performed several measurement cycles over a time period of several weeks or months. Due to this extended time period and the different measurement cycles performed at different time points, inter-assay variations between the different measurement cycles may occur with a high probability. Inter-assay variations may occur at certain steps, e.g. during the immunoprecipitation step (e.g. because of different lots of the precipitation antibody), or the Aβ(1-40) ELISA (due to different lots of the ELISA kits). Due to these inter-assay variations, the amount of the Aβ target peptides determined in the biological samples may not be comparable between the different measurement cycles and further, a statistical analysis of the amount of the Aβ target peptides, which includes all biological samples obtained in said multi-patient study, and the differentiation between AD patients and healthy subjects may become impossible.

To avoid said inter-assay variations and to make the results from different measurement cycles comparable and feasible for statistical analysis, the methods of the present invention are preferably performed in presence of an internal standard sample. Such an internal standard is for example an internal plasma standard—sample (ITS), which should be obtained from a well defined control subject, which is preferably a healthy subject. The concentration of the Aβ target peptides, such as Aβ(1-40) or Aβ(1-42), is constant in each ITS sample. Therefore, variations of the Aβ target peptide concentrations in ITS samples, which are found in different measurement cycles, reflect the inter-assay variability.

According to a preferred embodiment of the present invention, one or more ITS samples are included in each measurement cycle. After determining the level of the Aβ peptides in biological samples, such as plasma samples, the Aβ target peptide concentrations, such as the Aβ(1-40) or the Aβ(1-42) concentrations, of the biological samples are normalized to the concentration of the respective Aβ target peptide determined in the ITS sample(s), resulting in a relative Aβ target peptide level of each test sample.

In a yet preferred embodiment, the methods of the present invention comprise as further step the normalization of the Aβ target peptide concentrations, such as the Aβ(1-40) or the Aβ(1-42) concentrations, of the biological samples to the concentration of the respective Aβ target peptide determined in the ITS sample(s), resulting in a relative Aβ target peptide level of each biological sample.

As result, the comparison of the relative Aβ target peptide level in the biological samples determined in different measurement cycles (time point, antibody lot, ELISA lot) is more reliable than the comparison of the un-normalized Aβ target peptide levels.

The use of an ITS in the methods of the present invention is especially preferred for the determination of the concentration of Aβ(1-40) and/or Aβ(1-42), most preferably for the concentration of Aβ(1-40).

The methods of the present invention are not limited to plasma samples. If other body fluids (cerebrospinal fluid, urine, lymph, saliva, sudor, pleura fluid, synovial fluid, aqueous fluid, tear fluid, bile, pancreas secretion) shall be used in the methods of the present invention, these fluids have to be taken from a well defined control subject, preferably a healthy subject, as it was demonstrated herein for plasma samples. These fluids have then to be used as internal standard.

Thus, an internal standard sample according to the present invention is preferably selected from a plasma sample, cerebrospinal fluid sample, urine sample, lymph sample, saliva sample, sudor sample, pleura fluid sample, synovial fluid sample, aqueous fluid sample, tear fluid sample, bile sample, pancreas secretion sample. Most preferably, the internal standard sample is a plasma sample.

Suitably, the methods of the present invention are validated, wherein the subjects that are the donors of the biological samples, are well characterized in terms of the state of the neurodegenerative disease. Said characterization may be performed using conventional psychometric tests, such as DemTect, Mini-Mental-State Test, Clock-Drawing Test, ADAS-Cog (Alzheimer's Disease Assessment Scale-Cognitive), Blessed Test, CANTAB (Cambridge Neuropsychological Test Automated Battery), Cognistat (Neurobehavioral Cognitive Status Examination), Neuropsychiatric Inventory (NPI), Behavioral Pathology in Alzheimer's Disease Rating Scale (BEHAVE-AD), CERAD (The Consortium to Establish a Registry for Alzheimer's Disease) Clinical and Neuropsychological Tests, Cornell Scale for Depression in Dementia (CSDD), Geriatric Depression Scale (GDS) and the The 7 Minute Screen.

Possible antibodies for immuneprecipitation, which would be suitable in the present context are the following, although the present invention is not delimited to those specific working examples:
3D6, Epitope: 1-5 (Elan Pharmaceuticals, Innogenetics)
pAb-EL16, Epitope: 1-7
2H4, Epitope: 1-8 (Covance)
1E11, Epitope: 1-8 (Covance)
20.1, Epitope: 1-10 (Covance, Santa Cruz Biotechnology)
Rabbit Anti-Aβ Polyclonal Antibody, Epitope: 1-14 (Abcam)
AB10, Epitope: 1-16 (Chemicon/Upstate—part of Millipore)
82E1, Epitope: 1-16 (IBL)
pAb1-42, Epitope: 1-11
NAB228, Epitope: 1-11 (Covance, Sigma-Aldrich, Cell Signaling, Santa Cruz Biotechnology, Zymed/Invitrogen)
DE2, Epitope: 1-16 (Chemicon/Upstate—part of Millipore)
DE2B4, Epitope: 1-17 (Novus Biologicals, Abcam, Accurate, AbD Serotec)
6E10, Epitope: 1-17 (Signet Covance, Sigma-Aldrich)
10D5, Epitope: 3-7 (Elan Pharmaceuticals)
WO-2, Epitope: 4-10 (The Genetics Company)
1A3, Epitope 5-9 (Abbiotec)
pAb-EL21, Epitope 5-11
310-03, Epitope 5-16 (Abcam, Santa Cruz Biotechnology)
Chicken Anti-Human Aβ Polyclonal Antibody, Epitope 12-28 (Abcam)
Chicken Anti-Human Aβ Polyclonal Antibody, Epitope 25-35 (Abcam)
Rabbit Anti-Human Aβ Polyclonal Antibody, Epitope: N-terminal (ABR)
Rabbit Anti-Human Aβ Polyclonal Antibody (Anaspec)
12C3, Epitope 10-16 (Abbiotec, Santa Cruz Biotechnology)
16C9, Epitope 10-16 (Abbiotec, Santa Cruz Biotechnology)
19B8, Epitope 9-10 (Abbiotec, Santa Cruz Biotechnology)
pAb-EL26, Epitope: 11-26
BAM90.1, Epitope: 13-28 (Sigma-Aldrich)
Rabbit Anti-beta-Amyloid (pan) Polyclonal Antibody, Epitope: 15-30 (MBL)
22D12, Epitope: 18-21 (Santa Cruz Biotechnology)
266, Epitope: 16-24 (Elan Pharmaceuticals)
pAb-EL17; Epitope: 15-24
4G8, Epitope: 17-24 (Covance)
Rabbit Anti-Aβ Polyclonal Antibody, Epitope: 22-35 (Abcam)
G2-10; Epitope: 31-40 (The Genetics Company)
Rabbit Anti-Aβ, aa 32-40 Polyclonal Antibody (GenScript Corporation)
EP1876Y, Epitope: x-40 (Novus Biologicals)
G2-11, Epitope: 33-42 (The Genetics Company)
16C11, Epitope: 33-42 (Santa Cruz Biotechnology)
21F12, Epitope: 34-42 (Elan Pharmaceuticals, Innogenetics)
1A10, Epitope: 35-40 (IBL)
D-17 Goat anti-Aβ antibody, Epitope: C-terminal (Santa Cruz Biotechnology)

Particularly preferred antibodies for the immuneprecipitation are: 3D6 (Elan), BAN50 (Takeda), 82E1 (IBL), 6E10 (Covance), WO-2 (The Genectics Company), 266(Elan), BAM90.1 (Sigma), 4G8 (Covance), G2-10 (The Genetics Company), 1A10 (IBL), BA27 (Takeda), 11A5-B10 (Millipore), 12F4 (Millipore), 21F12 (Elan).

Particularly preferred antibody pairs for the immuneprecipitation are: 4G8 and 11A5-B10, 3D6 and 4G8, 6E10 and 4G8, 82E1 and 4G8, 4G8 and 12F4, 4G8 and 21F12, 3D6 and 21F12, 6E10 and 21F12, BAN50 and 4G8, 3D6 and 11A5-B10, 3D6 and 1A10, 3D6 and BA27, 6E10 and 11A5-B10, 6E10 and 1A10, 6E10 and BA27, 4G8 and 11A5-B10, 4G8 and 1A10, 4G8 and BA27, 4G8 and 12F4, 4G8 and 21F12.

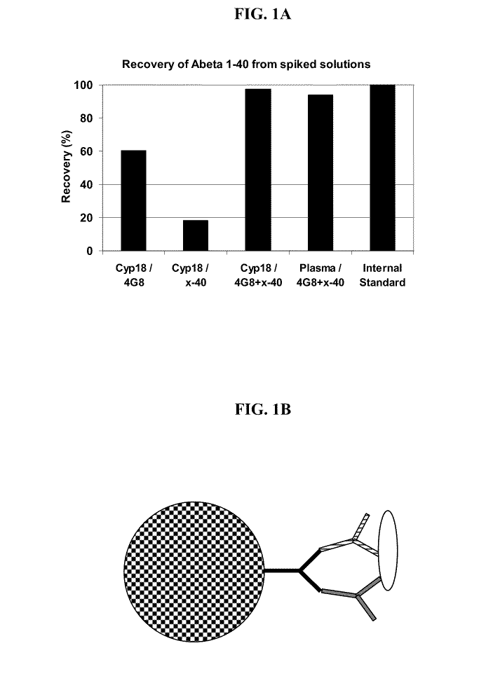

Apart from the above designated antibodies all other amyloid beta specific antibodies (monoclonal and polyclonal), which are suitable for immuneprecipitation can be used for the present inventive method (further suitable antibodies can e.g. be taken from www.alzforum.org). Decisive for good capture efficiency and thus constituting a key element of the present invention is the use of two, three or more different antibodies with different epitopes. The use of more than one antibody type for immuneprecipitation of Aβ peptides offers cooperative and surprisingly synergistic binding effects (avidity), which finally allows to achieve a tremendously higher capture efficiency (see FIG. 1).

The secondary antibodies in step ii) are specific against the host antibody type of the capture antibodies. Preferred secondary antibodies are anti-mouse antibodies and anti-rabbit antibodies.

After incubation of the complex with the magnetic beads in step iii), the beads may be washed with washing buffer (see examples of the present invention). Washing buffers, which contain detergents or other additives preventing unspecific binding, can be used for this step. Non-limiting examples for washing buffers are:
D-PBS containing 10 mg/ml Cyclophilin 18 (Cyp 18) and 0.05% Tween-20,
PBS+0.05% Tween-20,
TBS+0.05% Tween-20,
PBS+1% (w/v) BSA+0.05% Tween-20,
TBS+1% (w/v) BSA+0.05% Tween-20, and
Pierce ELISA Blocker (with Tween-20).

After elution of the immune complex from the beads in step iv), the solution is diluted in dilution buffer. Any dilution buffers, which can prevent unspecific interaction with surfaces and the immobilized first ELISA antibody can be used for this step. Non-limiting examples for dilution buffers are:
EIA buffer (dilution buffer of the IBL 1-40 (N) ELISA Kit),
PBS+1% (w/v) BSA+0.05% Tween-20,
TBS 30 1% (w/v) BSA+0.05% Tween-20, and
Pierce ELISA Blocker (with Tween-20).

ELISA-Kits that are able to quantify full length Aβ(1-40) are commercially available. Suitable ELISA-Kits for the quantification of Aβ(1-40) in the methods of the present invention are for example: Amyloid-β(1-40) (N) ELISA (IBL, JP27714); Aβ[1-40] Human ELISA Kit (Invitrogen); Human Amyloid beta (Amyloid-β), aa 1-40 ELISA Kit (Wako Chemicals USA, Inc.); Amyloid Beta 1-40 ELISA Kit (The Genetics Company).

ELISA-Kits that are able to quantify full length Aβ(1-42) are also commercially available. Suitable ELISA-Kits for the quantification of Aβ(1-42) in the methods of the present invention are for example: Amyloid-β(1-42) (N) ELISA (IBL, JP27712); Aβ[1-42] Human ELISA Kit (Invitrogen), Human Amyloid beta (Amyloid-β), aa 1-42 ELISA Kit (Wako Chemicals USA, Inc.), Amyloid Beta 1-40 ELISA Kit (The Genetics Company), INNOTEST® β-AMYLOID(1-42) (Innogenetics).

The inventive method is not limited to the exemplary aforementioned commercially available ELISA-Kits for Aβ(1-40) or Aβ(1-42). Numerous further sandwich ELISAs for full length Aβ(1-40) or Aβ(1-42) may be available in the prior art or may be developed by the skilled artisan. All these full length Aβ1-40 or Aβ1-42 sandwich ELISAs shall also be encompassed by the methods of the present invention and should typically comprise a suitable pair of capture and detection antibodies, which are specific for the complete N-terminus of Aβ(1-40) and/or Aβ(1-42) and the C-terminus ending at amino acid 40 or 42, respectively.

Such a full length Aβ(1-40) sandwich ELISA may comprise a first immobilized antibody recognizing specifically the C-terminus of Aβ(1-40) and a second labeled detection antibody recognizing specifically the complete N-terminus of Aβ(1-40).

A full length Aβ(1-42) sandwich ELISA may comprise a first immobilized antibody recognizing specifically the C-terminus of Aβ(1-42) and a second labeled detection antibody recognizing specifically the complete N-terminus of Aβ(1-42).

A full length Aβ(1-40) sandwich ELISA may also comprise a first immobilized antibody recognizing specifically the complete N-terminus of Aβ(1-40) and a second labeled detection antibody recognizing specifically the C-terminus of Aβ(1-40).

A full length Aβ(1-42) sandwich ELISA may also comprise a first immobilized antibody recognizing specifically the complete N-terminus of Aβ(1-42) and a second labeled detection antibody recognizing specifically the C-terminus of Aβ(1-42).

Suitable Aβ(1-40/42) N-terminal specific antibodies for use in the methods of the present invention are for example 3D6 (Elan), WO-2 (The Genetics Company), 82E1 (IBL), BAN-50 (Takeda). Numerous further Aβ(1-40/42) N-terminal specific antibodies may be available in the prior art or may be developed by the skilled artisan. All these Aβ(1-40/42) N-terminal specific antibodies are also encompassed by the methods of the present invention.

Suitable Aβ(1-40) C-terminal specific antibodies are for example G2-10 (The Genetics Company); 11A5-B10 (Millipore); 1A10 (IBL); BA27 (Takeda); EP1876Y (Novus Biologicals). Numerous further Aβ(1-40) C-terminal specific antibodies may be available in the prior art or may be developed by the skilled artisan. All these Aβ(1-40) C-terminal specific antibodies are also encompassed by the methods of the present invention.

Suitable Aβ(1-42) C-terminal specific antibodies are for example G2-11 (The Genetics Company); 12F4 (Millipore); Anti- Human Aβ(38-42) Rabbit IgG (IBL); 21F12 (Elan); BC05 (Takeda); 16C11 (Santa Cruz Biotechnology). Numerous further Aβ(1-42) C-terminal specific antibodies may be available in the prior art or may be developed by the skilled artisan. All these Aβ(1-42) C-terminal specific antibodies are also encompassed by the methods of the present invention.

According to a preferred embodiment, the detection antibodies are labelled.

For diagnostic applications, the detection antibody will typically be labelled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Gutigen et al., Ed., Wiley-Interscience. New York, N.Y. Pubs., (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra for example. Fluorescence can be quantified using a fluorimeter.

(c) Various enzyme-substrate labels are available. The enzyme generally catalyses a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g, firefly luciferase and bacterial luciferase; U.S. Pat. No, 4,737,456), luciferin, 2,3-dihydrophthalazinediones, malate dehydrogenase, urease, peroxidase such as horseradish peroxidase (HRPO), alkaline phosphatase. 0-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed Langone & H. Van Vunakis), Academic Press, New York, 73: 147-166 (1981).

Examples of enzyme-substrate combinations include, for example:
(i) Horseradish peroxidase (HRPO) with hydrogen peroxidase as a substrate, wherein the hydrogen peroxidase oxidizes a dye precursor (e.g. orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (β-D-Gal) with a chromogenic substrate (e.g. p-nitrophenyl-β-D-galactosidase) or the fluorogenic substrate 4-methylumbelliferyl-β-D-galactosidase.

Numerous other enzyme-substrate combinations are available to those skilled in the art.

(d) Another possible label for a detection antibody is a short nucleotide sequence. The concentration is then determined by a RT-PCR system (Imperacer™, Chimera Biotech).

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

The antibodies of the present invention may be employed in any known assay method, such as competitive binding assays, direct and indirect sandwich assays, and immuneprecipitation assays. Zola, Monoclonal Antibodies A Manual of Techniques, pp. 147-158 (CRC Press. Inc., 1987).

Competitive binding assays rely on the ability of a labeled standard to compete with the test sample analyte for binding with a limited amount of antibody. The amount of Aβ peptide in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

For the analysis of the Aβ(1-40) concentration in human all following body fluids can be used: blood, cerebrospinal fluid (CSF), urine, lymph, saliva, sweat, pleural fluid, synovial fluid, aqueous fluid, tear fluid, bile, pancreas secretion.

The novel method was established by the present inventors using blood samples (see the examples of the present invention). The present method is however not to be construed to be limited to blood samples. The method can also be employed using CSF, brain extract and urine samples, as well as all other human body fluids, e.g. the above mentioned in the same manner. Particularly preferred are plasma samples.

For immunohistochemistry analyses, the tissue sample may be fresh or frozen or may be embedded in paraffin and fixed with a preservative such as formalin, for example.

The method of the present invention is in step iv) not limited to a sandwich ELISA as quantification means. The method of the present invention encompasses also any other methods for quantification of an. Aβ target peptide, in particular Aβ(1-40), after the immuneprecipitation step. Suitable methods for the quantification of, for example Aβ(1-40), are:

1. Amyloid β1-40 HTRF® Assay (CisBio Bioassays):

The assay principle is based on TR-FRET, which is a combination of Time-Resolved Fluorescence and Förster Resonance Energy Transfer. Similar to the usual sandwich ELISA the Aβ(1-40) is bound by two antibodies; the antibodies are here, however, not bound on a surface, the interaction occurs in solution. Both antibodies are labeled with a fluorophor. When these two fluorophors are brought together by a biomolecular interaction a portion of energy captured by the donor fluorophor during excitation is transferred via FRET to an acceptor fluorophor, which will be excited as a result. The fluorescence of the acceptor fluorophor is measured. The measuring signal is correlated with the amount of FRET and thus, the amount of Aβ(1-40) in solution.

Similarly, based on a comparable principle, the Alphascreen™ Assay from Lilly can be used.

2. Multiplex Assay Systems

Multiplex Assay Systems are available from several manufacturers and are well known and broadly used in the field. A suitable example for use in the methods of the present invention is the INNO-BIA plasma Aβ forms assay (Innogenetics). This assay is a well standardized multiparameter bead-based immunoassay for the simultaneous quantification of human β-amyloid forms Aβ(1-42) and Aβ(1-40) or Aβ(X-42) and Aβ(X-40) in plasma using xMAP® technology (xMAβ is a registered trademark of Luminex Corp.).

This assay system is able to quantify up to 100 different analytes in parallel. The basis of this method are small spherical polystyrol particles, called microspheres or beads. In analogy to ELISA and Western Blot these beads serve as a solid phase for the biochemical detection. These beads are color-coded, so that 100 different bead classes can be distinguished. Every bead class has one specific antibody (e.g. against Aβ(1-40)) immobilized on the microsphere surface. If the Aβ(1-40) concentration increases more peptide molecules will be bound by the beads of this class. The detection of the binding of the analyte is carried out by a second anti-Aβ(1-40) antibody, which is labeled with another fluorescence dye, emitting green light. The sample is handled comparable to FACS analysis. The microspheres are singularized by hydrodynamic focusing and analyzed by laser-based detection system, which can make a quantification on the basis of the green fluorescence and identify the bound analyte by the specific coloration of the bead. Thus, it is possible to determine the concentration of multiple analytes in one sample.

3. Quantification by Mass Spectrometry
For quantification of Aβ(1-40) also the SELDI-TOF mass spectrometry was used (Simonsen et al., 2007 (2)).
Quantitative analysis of Aβ peptides using immuneprecipitation and MALDI-TOF mass spectrometry. ¹⁵N labeled standard Aβ peptides are used for calibration. (Gelfanova et al., 2007).

4. Western Blot Analysis
2D-Gel electrophoresis coupled with Western Blot analysis may be a suitable method to quantify Aβ peptides (Sergeant et al., 2003; Casas et al., 2004).

Diagnostic Kits

As a matter of convenience, the antibodies of the present invention can be provided in a kit, i.e., a packaged combination of reagents in predetermined amounts with instructions for performing the diagnostic assay. Where the antibody is labelled with an enzyme, the kit will include substrates and cofactors required by the enzyme (e.g. a substrate precursor which provides the detectable chromophore or fluorophore). In addition, other additives may be included such as stabilizers, buffers (e.g. a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

The diagnostic kit of the invention is especially useful for the detection and diagnosis of amyloid-associated diseases and conditions, preferably Alzheimer's disease.

Uses

The method of the present invention makes it possible for the first time to detect and quantify Aβ peptides, in particular Aβ(1-40), or a functional equivalent thereof, in a reliable manner. In particular, the present invention provides Aβ(1-40) as a plasma biomarker, which is suitable for a differential diagnosis of Alzheimer's disease, in particular in the early stages of the disease.

Therefore, in one embodiment, the invention is directed to the use of method for the detection of an Aβ target peptide for the diagnosis of Alzheimer's disease, preferably the differential diagnosis of Alzheimer's disease, in particular in the early stages of the disease. Preferably, the early stage of Azheimer's disease is Mild Cognitive impairment.

In a further embodiment, the invention is directed to the use of the Aβ target peptides for the diagnosis of Alzheimer's diseases, preferably the differential diagnosis of Alzheimer's disease, in particular in the early stages of the disease. Preferably, the early stage of Azheimer's disease is Mild Cognitive impairment.

In particular, it is preferred that the Aβ target peptide, which shall be used for diagnosis of Alzheimer's disease, is detected and quantified with a method according to the present invention.

In a preferred embodiment, the Aβ target peptide is Aβ(1-40) or a functional equivalent thereof.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the invention are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

The invention embraces all combinations of preferred and more preferred groups and embodiments of groups recited herein.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

1. Materials and Methods

1.1 Patients and Healthy Controls

Patients with a clinical diagnosis of AD and healthy controls were recruited. In a prestudy examination the neuropsychological functions of all participants of the study were tested by several psychometric tests (DemTect, Mini-Mental-State Test, Clock-drawing test)

DemTect Test

The DemTect scale is a brief screening test for dementia comprising five short subtests (10-word list repetition, number transcoding, semantic word fluency task, backward digit span, delayed word list recall) (Kessler et al., 2000). The raw scores are transformed to give age- and education-independent scores, classified as ‘suspected dementia’ (score≦8), ‘mild cognitive impairment’ (score 9-12), and ‘appropriate for age’ (score 13-18).

MMSE

The Mini-Mental State Examination (MMSE) or Folstein test is a brief 30-point questionnaire test that is used to assess cognition. It is commonly used in medicine to screen for dementia. In the time span of about 10 minutes it samples various functions including arithmetic, memory and orientation. It was introduced by Folstein et al., 1975, and is widely used with small modifications.

The MMSE includes simple questions and problems in a number of areas: the time and place of the test, repeating lists of words, arithmetic, language use and comprehension, and basic motor skills. For example, one question asks to copy a drawing of two pentagons (see table 1). Any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing.

Clock-Drawing Test

Scoring of the clocks was based on a modification of the scale used by Shulmann et al., 1986. All circles were predrawn and the instruction to the subjects was to “set the time 10 after 11”. The scoring system (see table 2) ranges in scores from 1 to 6 with higher scores reflecting a greater number of errors and more impairment. This scoring system is empirically derived and modified on the basis of clinical practice. Of necessity, it leaves considerable room for individual judgment, but it is simple enough to have a high level of inter-rater reliability. Our study lends itself to the analysis of the three major components. These include cross-sectional comparisons of the clock-drawing test with other measures of cognitive function; a longitudinal description of the clock-drawing test over time, and the relationship between deterioration on the clock-drawing test and the decisions to institutionalize.

TABLE 1
Typical Mini-Mental State Examination

| | | Max | |
|---|---|---|---|
| Section | Questions | Points | Score |
| 1) Orientation | a) Can you tell me today's | 5 | |
| | (date)/(month)/(year)? | | |
| | Which day is it today? | | |
| | Can you tell me which (season) it is? | | |
| | b) What town/city are we in? | | |
| | What is the (county)! (country)? | | |
| | What (building) are we in and on | 5 | |
| | what (floor) | | |
| 2) Registration | I should like to test your memory. | 3 | |
| | (name three common objects: | | |
| | “ball, car, man”) | | |
| | Can you repeat the words I said? | | |
| | (1 point per word) | | |
| | (repeat up to 6 trials until all three are | | |
| | remembered) | | |
| 3) Attention | a) From 100 keep subtracting 7 and | 5 | |
| and | give each answer. Stop after 5 answers. | | |
| Calculation | (93-86-79-72-65) | | |
| | Alternatively: | | |
| | b) Spell the word “World” backwards. | | |
| | (D_ L_ R_ O_ W) | | |
| 4) Recall | What were the three words I asked | 3 | |
| | you to say earlier? | | |
| | (skip this test if all of these objects were not | | |
| | remembered during the registration test) | | |
| 5) Language | Name the following objects (show a watch) | 2 | |
| Naming | and (show a pencil) | | |
| Repeating | Repeat the following: “no ifs, ands or buts” | 1 | |
| 6) Reading | (show card or write: “Close your Eyes”) | 1 | |
| | Read this sentence and do what it says. | | |
| Writing | Now can you write a short sentence for me? | 1 | |
| 7) Three stage | (present paper) | 3 | |
| command | Take this paper in you left (or right) hand, | | |
| | fold it in half, and place it on the floor. | | |
| 8) Construction | Will you copy this drawing please? | 1 | |
| | 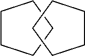 | | |
| Total score | | 30 | |

TABLE 2
Typical Clock-drawing test

| | |
|---|---|
| 1. Perfect | 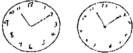 |
| 2. Minor visuospatial errors Examples Mildly impaired spacing of times Draws times outside circle Turns page while writing numbers so that some numbers appear upside down Draws in linies (spokes) to orient spacing | 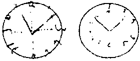 |
| 3. Inaccurate representation of 10 after 11 when visuosptial organization is perfect or shows only minor deviations Examples Minute hand points to 10 Writes ‘10 after 11’ Unable to make any denotation of time | 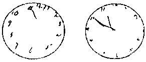 |
| 4. Moderate visuospatial disoganization of times such that accurate denotation of 10 after 11 is impossible Example Moderately poor spacing Omits numbers Perseveration-repeats circle or continues on past 12 to 13, 14, 15, etc Right-left reversal-numbers drawn counter clockwise Dysgraphia-unable to write numbers accurately | 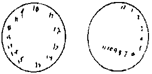 |
| 5. Severe level of disorganization as described in 4 | 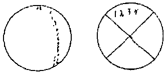 |
| 6. No reasonable representation of a clock Exclude severe depression or other psychotic states Examples No attempt at all No semblance of a clock at all Writes a word or name | 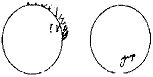 |

1.2 Blood Samples

1.2.1 Test Samples

After prestudy examination the study started 2 weeks later with blood withdrawal from all participants.

All blood samples for the determination of AD biomarkers were collected into three polypropylene tubes, respectively:
1. containing potassium-EDTA (Sarstedt Monovette, 02.1066.001) for EDTA plasma
2. containing Li-heparine (Sartstedt Monovette, 02.1065.001) for heparine plasma
3. blank (Sarstedt Monovette, 02.1063.001) for serum

All samples were collected by venous puncture or by repeated withdrawal out of an inserted forearm vein indwelling cannula. Blood was collected according to the selected time schedule (as described in chapter 1.1 above). It was centrifuged with 1550 g (3000 rpm) for 10 min at 4° C. to provide plasma. Plasma or serum of each separate sample was pipetted off, filled in one 5 ml polypropylene cryo-tube (Carl-Roth, E295.1) and stored frozen at −80° C. Samples were centrifuged within one hour after blood withdrawal.

1.2.2 Samples to Establish an Internal EDTA Plasma Standard—Control (ITS)

A blood sample (45 ml) was taken from a well defined control person by venous puncture or by repeated withdrawal out of an inserted forearm vein indwelling cannula into five polypropylene tubes containing potassium-EDTA (Sarstedt Monovette, 02.1066.001) for EDTA plasma. All five tubes were centrifuged with 1550 g (3000 rpm) for 10 min at 4° C. to provide plasma. Plasma was transferred to 2 ml polypropylene tubes (Eppendorf, 0030120.094) to 1 ml aliquots. The aliquots of this internal plasma standard were stored at −80° C. The sample was centrifuged within one hour after blood withdrawal. The control plasma was labeled as “Internal EDTA plasma standard—control” (ITS). If other body fluids (cerebrospinal fluid, urine, lymph, saliva, sudor, pleura fluid, synovial fluid, aqueous fluid, tear fluid, bile, pancreas secretion) shall be used in the methods of the present invention, these fluids have to be taken from a well defined control person, as it was demonstrated herein for plasma samples.

1.3 Laboratory Methods

1.3.1 Test Samples

Immuneprecipitation

EDTA plasma samples (containing 4 ml plasma) (whereby the invention is not limited to EDTA plasma; e.g. heparin plasma, or serum can also be used) were thawed and aliquoted a 1 ml in 2 ml polypropylene tubes (Eppendorf, 0030120.094). One pill of protease inhibitor (Roche, Complete mini Protease inhibitor cocktail, 11836153001) was dissolved in 1 ml D-PBS (Invitrogen, 14190-094). 25 μl of the protease inhibitor solution was added to 1 ml EDTA plasma. All aliquots were frozen and stored again at −80° C., except one tube of each sample. These remaining tubes of plasma samples were spiked with 10 μl of 10% Tween-20. To each tube 2.5 μg anti-amyloid β(17-24) antibody 4G8 (Millipore, MAB1561), 2.5 μg anti-amyloid β(x-42) antibody 12F4 (Millipore, 05-831) and 2.5 μg anti-amyloid β(x-40) antibody 11A5-B10 (Millipore, 05-799) were added.

All plasma tubes were incubated overnight at 4° C. in an overhead shaker. For immobilization of the amyloid β-antibody complex 100 μl anti-mouse magnetic beads (Invitrogen, 112-02D) were used for each 1 ml plasma sample. Instead of these special anti-mouse antibodies conjugated on magnetic beads all other anti-mouse antibodies or anti-host antibodies (host: origin of primary antibodies listed above) can be used. These antibodies can be immobilized on several matrices (column matrices and bead matrices) via different conjugation strategies, non-limiting examples being Biotin-Streptavidin interaction, tosyl-activated surface, epoxy-activated surface, amine-surface, or a carboxylic surface. Before use 100 μl beads were transferred from the original bottle into a 2 ml tube and washed 3 times with 1 ml PBS. After washing the beads were re-suspended in 200 μl PBS. The plasma tubes were shortly (30 sec) centrifuged with 2000×g. The supernatants were transferred into the tubes containing the anti-mouse magnetic beads. The tubes were incubated overnight at 4° C. in an overhead shaker.
On the next day the tubes were put into a magnetic separator to allow the beads to be drawn to the tube wall. After about one minute the supernatant was carefully removed and the beads were washed twice with 500 μl D-PBS containing 10 mg/ml Cyclophilin 18 and 0.05% Tween-20.

Elution of Captured Amyloid β

After the last washing step the solution was drawn out, the tubes were taken from the magnetic separator and 100 μl 50% (v/v) methanol/0.5% (v/v) formic acid were added to each tube and the beads were re-suspended by slight shaking. All tubes were incubated for 1 hour at room temperature. Afterwards the tubes were again placed in the magnetic separator and 40 μl from each tube were mixed with 440 μl EIA buffer (dilution buffer of the IBL 1-40 (N) ELISA Kit). The pH of the diluted sample was adjusted with 16 μl 400 mM Na₂HPO₄, 400 mM KH₂PO₄ pH 8.0 to the pH of the EIA buffer.

Quantification of the Eluted Amyloid β Peptides

The determination of the peptide concentration was performed using the IBL 1-40(N) ELISA Kit (IBL, JP27714).

Instead of the above described Aβ(1-40) ELISA all other commercially available ELISA kits, which are able to detect full length Aβ-40 can be used.

The diluted samples were applied to the ELISA plate (100 μl per Well, repeat determination). The ELISA standard were taken from the Kit, dissolved and diluted according to the manufacturer's instruction protocol. After application of all samples and concentration standards the ELISA plate was incubated for 18 h at 4° C. On the next day the ELISA was developed according to the manufacturer's instruction protocol.

After stopping the colorimetric reaction the absorbance in each well was determined at 450 nm corrected by absorbance at 550 nm using a plate reader (TECAN Sunrise).

The determination of the standard curve was carried out by plotting the corrected absorbance at 450 nm versus the corresponding standard peptide concentration. The curve was fitted with the four-parameter equation (Equation. 1) using Origin 7.0 (Microcal).

$$y = \frac{A 1 - A 2}{1 + {( \frac{x}{{x}_{0}} )}^{p}} + A 2 , \qquad Equation 1$$

y represents the measured absorbance and x the corresponding concentration, A1-lower asymptote, A2-upper asymptote.

The calculation of the Aβ(1-40) concentrations on ELISA of each sample occurred based on the according absorbance value using Equation. 2.

$$x = {x}_{0} \cdot \sqrt[p]{\frac{A 1 - y}{y - A 2}} \qquad Equation 2$$

To determine the concentration in the plasma sample the calculated concentration was corrected by the EIA buffer dilution (including pH adjustment), factor 12.4, and the concentration effect (1 ml to 100 μl) of the immuneprecipitation by factor 0.1. The determined plasma Aβ(1-40) concentrations were denoted in pg/ml.

Statistical Analysis

The correlation of the plasma concentration of Aβ(1-40) with the existence of a positive clinical diagnosis of Alzheimer's disease was examined using the Student's t-test.

1.3.2 Internal EDTA Plasma Standard—Control (ITS) Samples

Immuneprecipitation

The immunoprecipitation of the ITS sample was in general performed according to the same method as used for the test samples described above.

The ITS sample (containing 1 ml EDTA plasma) (heparin plasma, serum also possible) and the test samples were thawed at the same time. One pill of protease inhibitor (Roche, Complete mini Protease inhibitor cocktail, 11836153001) was solved in 1 ml D-PBS (Invitrogen, 14190-094). 25 μl of the protease inhibitor solution was added to 1 ml ITS sample. According to the method and handling of the test samples, the ITS plasma sample was spiked with 10 μl of 10% Tween-20, 2.5 μg anti-amyloid β(17-24) antibody 4G8 (Millipore, MAB1561), 2.5 μg anti-amyloid β(x-42) antibody 12F4 (Millipore, 05-831) and 2.5 μg anti-amyloid β(x-40) antibody 11A5-B10 (Millipore, 05-799).

All following steps were performed according to the same method as used for the test samples described above.

Elution of Captured Amyloid β

The ITS sample was treated according to the same method as used for the test samples described above.

Quantification of the Eluted Amyloid β Peptides

The quantification of the eluted amyloid β peptides in the ITS sample was performed according to the same method as used for the test samples described above.

Statistical Analysis

Within one measurement the Aβ(1-40) concentration of the ITS sample and the test plasma samples were determined together on one ELISA plate. Thereafter, the determined plasma Aβ(1-40) concentrations of the test samples were normalized to the determined concentration of the ITS sample according to Equation 3:

$$relative A β ( 1 \text{-} 40 ) level = \frac{\begin{matrix}Plasma A β ( 1 \text{-} 40 ) concentration \\ in test sample\end{matrix}}{\begin{matrix}Plasma A β ( 1 \text{-} 40 ) \\ concentration in ITS sample\end{matrix}} \qquad Equation 3$$

2. Results

2.1 Demographic Characteristics

Overall, 45 persons have participated in the present study, 30 healthy controls and 15 AD patients (the AD patients being designated as “patients” in the following). To observe possible influences of age on plasma Aβ, control persons were selected over a wide range of age and subclassified into three groups. Group I contains subjects with an age of 18 to 30, Group II those with an age from 31 to 45 and Group III subjects with an age from 46 to 65. The demographic characteristics are shown in Table 3.

TABLE 3
Demographic Characteristics Healthy controls

| | Group I | Group II | Group III | |
|---|---|---|---|---|
| | (18-30) | (31-45) | (46-65) | AD patients |
| No. | 10 | 10 | 10 | 15 |
| Age at | 25.8 ± 2.9 | 38.4 ± 4.7 | 54 ± 6.9 | 79.13 ± 7.09 |
| baseline | | | | |
| (mean ± | | | | |
| SDEV), | | | | |
| Height, cm | 175.5 ± 11.6 | 175.1 ± 7.2 | 167.5 ± 10.9 | 168.4 ± 10.34 |
| (mean ± | | | | |
| SDEV) | | | | |
| Weight, kg | 71.33 ± 11.8 | 71.36 ± 13.5 | 75.81 ± 13.3 | 72.00 ± 12.31 |
| (mean ± | | | | |
| SDEV) | | | | |
| Sex (% | 50 | 50 | 50 | 40 |
| women) | | | | |

2.2 Psychometric Tests

For evaluation of the neuropsychological functions all participants have performed the DemTect, Mini-Mental-State Test and Clock-Drawing test, as described above. These tests have been made in prestudy, as well as 3 months, 6 months, 9 months and 12 months after start of the study.

DemTect Test

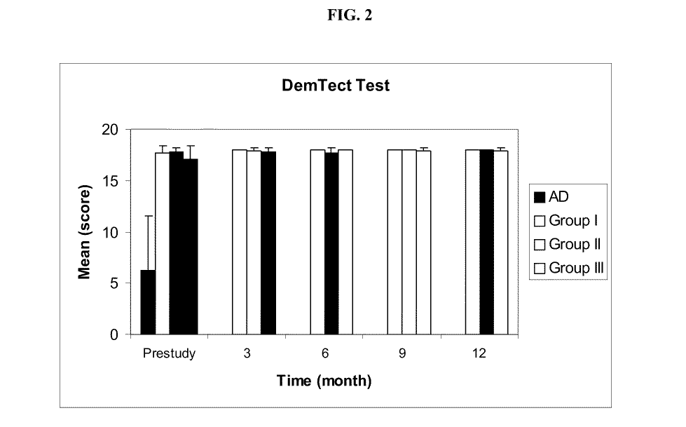

The raw scores are transformed to give age- and education-independent scores, classified as ‘suspected dementia’ (score≦8), ‘mild cognitive impairment’ (score 9-12), and ‘appropriate for age’ (score 13-18). The test results for all visits are shown in FIG. 2.

There are clear differences between the three groups of healthy subjects compared with the patients. In particular, it can be seen that the mean score of AD patients is less than half the mean score of the healthy controls. The patient's mean has not changed over time and is thus given only once in FIG. 2.

Mini-Mental-State Test

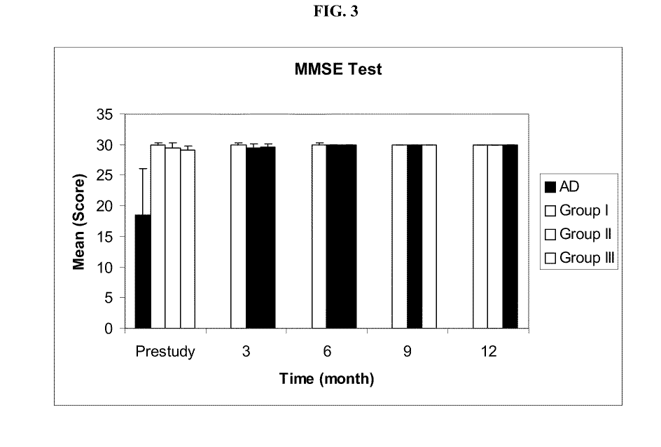

In the MMS Test any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing. The test results are shown in FIG. 3. The same healthy controls and AD patients participated in this test.

There are clear differences between the three groups of healthy subjects compared with the patients, which are shown in FIG. 3 in a similar fashion as in FIG. 2.

Clock-Drawing Test

The scoring system of the Clock Drawing Test ranges in scores from 1 to 6 with higher scores reflecting a greater number of errors and more impairment. This scoring system is empirically derived and modified on the basis of clinical practice. Of necessity, it leaves considerable scope for individual judgment, but it is simple enough to have a high level of inter-rater reliability. Again, the same group of healthy controls and AD patients participated as in the two tests above.

The present study lends itself to the analysis of the following three major components. These include cross-sectional comparisons of the clock-drawing test with other measures of cognitive function; a longitudinal description of the clock-drawing test over time, and the relationship between deterioration on the clock-drawing test and the decisions to institutionalize.

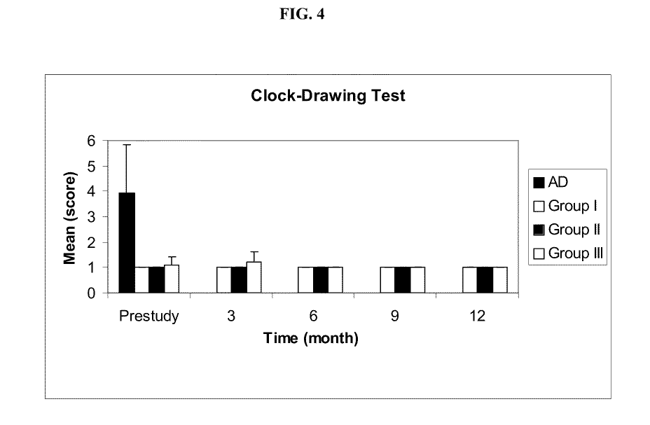

The test results are shown in FIG. 4.

There are clear differences between the three groups of healthy subjects compared with the patients. The results are depicted in FIG. 4 in a similar fashion of for FIGS. 2 and 3.

2.3 Plasma Aβ(1-40) Concentration, not Normalized

The Aβ(1-40) concentration was determined in EDTA plasma of the T0+9 months series, as described above. Further samples of the T0+9 series were used to optimize and establish the new immuneprecipitation method. Overall, the final optimized method was tested with 10 AD samples and 26 control samples. The determined plasma Aβ(1-40) concentrations are shown in Table 4.

TABLE 4
Plasma 4β(1-40) concentrations of all analyzed samples (T0 + 9 months series)
The mean concentration values and the standard error of mean for all
four groups were calculated. The Student's t-test has compared
the AD group with each single control group.

| AD Group | | Control Group 18-30 | | Control Group 31-45 | | Control Group 46-65 | |
|---|---|---|---|---|---|---|---|
| Subject | Aβ(1-40) (pg/ml) | Subject | Aβ(1-40) (pg/ml) | Subject | Aβ(1-40) (pg/ml) | Subject | Aβ(1-40) (pg/ml) |
| Nr. 10 | 366.86 | Nr. 09 | 320.46 | Nr. 13 | 291.76 | Nr. 02 | 337.81 |
| Nr. 11 | 364.57 | Nr. 27 | 302.80 | Nr. 15 | 296.74 | Nr. 08 | 257.64 |
| Nr. 14 | 424.75 | Nr. 32 | 237.59 | Nr. 17 | 320.36 | Nr. 12 | 287.10 |
| Nr. 16 | 347.81 | Nr. 36 | 239.33 | Nr. 18 | 331.72 | Nr. 19 | 317.78 |
| Nr. 20 | 403.27 | Nr. 37 | 326.98 | Nr. 25 | 297.93 | Nr. 21 | 288.38 |
| Nr. 22 | 262.96 | Nr. 40 | 330.16 | Nr. 28 | 250.21 | Nr. 23 | 426.33 |
| Nr. 26 | 463.03 | Nr. 41 | 280.66 | Nr. 29 | 268.31 | Nr. 24 | 356.83 |
| Nr. 30 | 423.09 | Nr. 42 | 359.67 | Nr. 31 | 349.74 | Nr. 33 | 277.00 |
| Nr. 39 | 402.74 | Nr. 44 | 319.90 | Nr. 38 | 245.71 | | |
| Nr. 45 | 391.88 | | | | | | |
| Mean | 385.10 | Mean | 301.95 | Mean | 294.72 | Mean | 318.61 |
| SEM | 17.27 | SEM | 13.91 | SEM | 11.88 | SEM | 19.27 |

| | | | |
|---|---|---|---|
| T-test AD Group vs. Control Group | 0.00180 | 0.00058 | 0.02059 |

For all control groups, a significant difference between the healthy volunteers and the AD group was obtained. Upon closer review of the individual concentrations within one group two subjects were eye-catching. The AD subject Nr. 22 shows an Aβ(1-40) concentration, which is typically for healthy controls. It does not fit into the AD group. If the individual results of the prestudy psychometric tests (see FIGS. 2-4 and table 4 below) are compared, it becomes evident that subject No. 22 has the highest score of all those analyzed participants by far, which were categorized into the AD group. The DemTect score is in the range of ‘appropriate for age’ and the MMSE score is on the upper limit of the range for MCI subjects. In contrast, the control subject No. 23 shows a, Aβ(1-40) concentration typically for the AD group. The prestudy psychometric tests (see FIGS. 2-4 and table 5 below) offer, that No. 23 has the lowest score of all those analyzed participants by far, which were categorized into control groups. It is evident that the increased plasma Aβ(1-40) level is the first indication for the onset of Alzheimer's disease.

TABLE 5
Psychometric tests (prestudy) of all analyzed subjects

| | | Mini-Mental- | Clock- |
|---|---|---|---|
| | DemTect | State | Drawing |
| AD group | | | |
| Nr. 10 | 1 | 12 | 6 |
| Nr. 11 | 5 | 18 | 5 |
| Nr. 14 | 6 | 22 | 4 |
| Nr. 16 | 1 | 11 | 5 |
| Nr. 20 | 10 | 19 | 2 |
| Nr. 22 | 16 | 26 | 2 |
| Nr. 26 | 8 | 25 | 6 |
| Nr. 30 | 0 | 0 | 6 |
| Nr. 39 | 4 | 21 | 5 |
| Nr. 45 | 9 | 25 | 1 |
| Control group 18-30 | | | |
| Nr. 09 | 16 | 30 | 1 |
| Nr. 27 | 18 | 30 | 1 |
| Nr. 32 | 18 | 30 | 1 |
| Nr. 36 | 18 | 30 | 1 |
| Nr. 37 | 17 | 30 | 1 |
| Nr. 40 | 18 | 30 | 1 |
| Nr. 41 | 18 | 30 | 1 |
| Nr. 42 | 18 | 29 | 1 |
| Nr. 44 | 18 | 30 | 1 |
| Control Group 31-45 | | | |
| Nr. 13 | 17 | 29 | 1 |
| Nr. 15 | 18 | 29 | 1 |
| Nr. 17 | 18 | 29 | 1 |
| Nr. 18 | 18 | 28 | 1 |
| Nr. 25 | 17 | 30 | 1 |
| Nr. 28 | 18 | 30 | 1 |
| Nr. 29 | 18 | 30 | 1 |
| Nr. 31 | 18 | 30 | 1 |
| Nr. 38 | 18 | 30 | 1 |
| Control Group 46-65 | | | |
| Nr. 02 | 17 | 29 | 1 |
| Nr. 08 | 18 | 30 | 1 |
| Nr. 12 | 18 | 30 | 1 |
| Nr. 19 | 18 | 28 | 1 |
| Nr. 21 | 18 | 29 | 1 |
| Nr. 23 | 14 | 28 | 2 |
| Nr. 24 | 16 | 29 | 1 |
| Nr. 33 | 17 | 29 | 1 |

Based on the findings of the psychometric tests and plasma analysis it cannot be assumed that AD subject No. 22 is correctly categorized. In the case of control subject No. 23 a possible early stage of the Alzheimer's Disease is conceivable. Therefore, the data were statistically analyzed including or excluding these two subjects (Table 6 below).

TABLE 6
Statistical analysis of analyzed AD subjects vs. controls
(Groups I-III together)

| | AD Group | All controls | AD Group | All Controls |
|---|---|---|---|---|
| | (all analyzed | (all analyzed | (except | (except |
| | samples) | samples) | No. 22) | No. 23) |
| Mean | 385.10 pg/ml | 304.57 pg/ml | 398.67 pg/ml | 299.70 pg/ml |
| Median | 397.31 pg/ml | 300.36 pg/ml | 402.74 pg/ml | 297.93 pg/ml |
| SDEV | 54.61 | 43.54 | 35.85 | 36.50 |
| SEM | 17.27 | 8.54 | 11.94 | 7.30 |

| | | |
|---|---|---|
| T-Test | p = 5.14E−05 | p = 6.09E−08 |

To enable an early clinical diagnosis, preferably at a stage where further symptoms are not yet available, it is important that the biomarker to be used is altered already in early stages of Alzheimer, for example during Mild Cognitive Impairment (MCI). This is particularly important if early onset therapy is necessary to prolong life and life's quality for an individual.

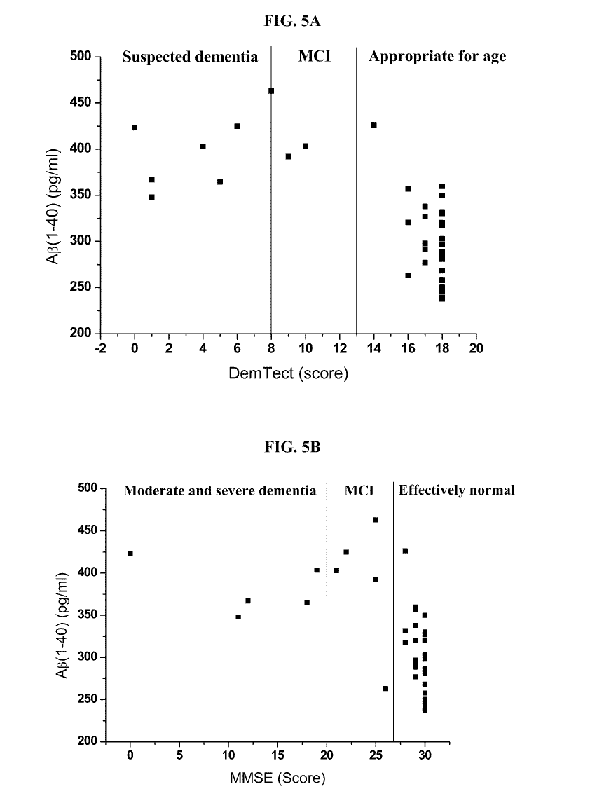

To determine whether the Aβ(1-40) level would be suitable as an early onset marker for AD the association of plasma concentration of Aβ(1-40) with the DemTect and MMSE score (FIG. 5) has been evaluated in further experiments.

As expected from the results of the present inventor's studies above, the lowest Aβ(1-40) concentrations were observed in control subjects corresponding with a high DemTect and

MMSE score, respectively. The highest concentrations were observed in persons which are categorized with mild cognitive impairment. A further decline of the scores, indicating moderate or severe dementia, shows also a decrease of plasma Aβ(1-40) level which is located between effectively normal and MCI. This finding indicates that a significantly increased level of plasma Aβ(1-40) is an initial and early marker for the onset of early stages of Alzheimer, at which point in time no or only a minor decline of cognitive functions is observable

2.4 Plasma Aβ(1-40) Concentration, Normalized After Inclusion of the ITS Samples

The comparison of relative Aβ(1-40) concentration was performed in a second series of measurements (T0+6 months) of EDTA plasma samples. In a first step, test samples from control subjects and AD patients were analyzed together on one ELISA plate. The determined plasma Aβ(1-40) levels were normalized to the mean value of all samples from the control subjects, which were analyzed within this measurement cycle. The normalization of the Aβ levels was performed according to equation 4:

$$relative A β ( 1 \text{-} 40 ) level = \frac{\begin{matrix}Plasma A β ( 1 \text{-} 40 ) concentration \\ in AD test sample\end{matrix}}{\begin{matrix}Meanplasma A β ( 1 \text{-} 40 ) \\ concentration in control samples\end{matrix}} \qquad Equation 4$$

Overall 10 control and 10 AD samples were analyzed in two different measurement cycles. The relative Aβ(1-40) concentrations are shown in table 7.

TABLE 7
Relative plasma Aβ(1-40) concentration of 20
analyzed samples (T0 + 6 month series)

| AD group | | Control group | |
|---|---|---|---|
| | relative Aβ(1-40) | | relative Aβ(1-40) |
| Subject | level | Subject | level |
| Nr. 11 | 1.22 | Nr. 6 | 1.16 |
| Nr. 14 | 1.27 | Nr. 7 | 1.09 |
| Nr. 16 | 1.11 | Nr. 8 | 0.84 |
| Nr. 20 | 1.72 | Nr. 9 | 1.02 |
| Nr. 22 | 0.92 | Nr. 12 | 0.90 |
| Nr. 26 | 1.44 | Nr. 13 | 1.05 |
| Nr. 30 | 1.55 | Nr. 15 | 0.93 |
| Nr. 39 | 1.47 | Nr. 17 | 0.95 |
| Nr. 43 | 1.13 | Nr. 18 | 1.01 |
| Nr. 45 | 1.36 | Nr. 19 | 1.05 |
| Mean | 1.32 | Mean | 1.00 |

| | |
|---|---|
| T-Test AD group vs. Control group | 9.3e−4 |

As shown in table 7, the use of an internal standard within every measurement cycle improves the reliability and enables a better comparison between the values for the Aβ peptide levels determined in different measurement cycles. However, in blinded studies, it is not possible to differentiate between AD and control samples. Therefore the use of an internal plasma standard (ITS), which is co-analyzed in every measurement cycle is the only way to compare Aβ peptide values from different measurements cycles.

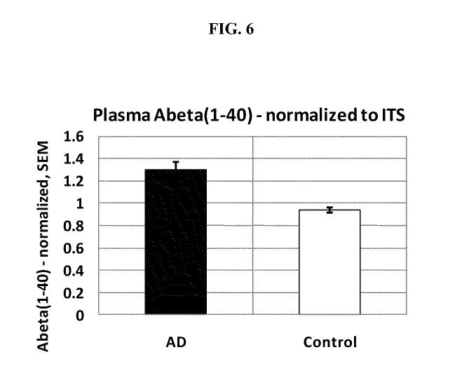

Accordingly, the plasma Aβ(1-40) levels of the T0+6 months samples was analyzed in presence of the ITS. All values were normalized to the Aβ(1-40) concentration of the ITS. The result is shown in FIG. 6. As shown in FIG. 6, the mean value of the relative Aβ(1-40) level is 1.31 for all analyzed AD samples and 0.94 for all analyzed samples from healthy control subjects. The comparison of these values with the mean values in table 7 shows a very good consistency concerning the increase of the plasma Aβ(1-40) level by about 32% in AD patients compared to healthy controls. These data indicate, that the use of an internal plasma standard analyzed together with unknown plasma samples within every measurement cycle increases the reliability and comparability of the determined Aβ(1-40) levels, which were determined in different measurement cycles.

3. Discussion

The present inventors could show that high plasma concentrations of Aβ(1-40) were associated with a positive clinical diagnosis of Alzheimer's Disease. Although earlier studies (van Oijen et al., 2006; Mayeux et al., 2003; Mehta et al., 2000,) made attempts to show this correlation, the statistical significance was not convincing which lead to the belief that Aβ(1-40) would not be suitable as marker for AD, both as no statistically significant correlation could be established and in view of the lack of a suitable method for determination. In the present studies, the Aβ(1-40) concentrations were directly determined by a double-antibody determination method. In the Rotterdam study (van Oijen et al., 2006), the mean concentration value of all samples was 192.0 pg/ml. Mayeux and co-workers found 153.6 pg/ml in AD at baseline and 133.3 pg/ml in non-demented elderly, Mehta and co-workers obtained 272 pg/ml and 219 pg/ml in patients with sporadic AD and in healthy controls, respectively. In the present study, the inventors obtained mean plasma Aβ(1-40) concentrations of 385 pg/ml (AD patients) and 304 pg/ml (healthy controls), respectively. The increase of detected plasma Aβ(1-40) is the result of the use of the novel bivalent capture system. As explained above in detail in this system one Aβ peptide molecule is bound by two antibody molecules recognizing two different epitopes. The first (capture) antibody interacts with amino acids 17-24 of Aβ (1-40). The second capture antibody binds to the C-terminus of Aβ(1-40). Both antibodies were immobilized in a preferred embodiment by one anti-mouse antibody on magnetic beads. Without the wish to be bound to this hypothesis it is assumed that a cooperative binding between the two capture antibodies and the Aβ peptides in human plasma can be achieved. This binding, which has proven to be particularly strong and specific, provides for the capturing of all Aβ(1-40) peptide molecules from a given sample, for example plasma and further ensures the removal of other plasma proteins which would disturb the quantification via ELISA. Significant differences between AD patients and controls became evident with the present study wherein Aβ(1-40) peptide molecules of a given sample can be detected in a quantitative manner.

In conventional methods for the prediction of Alzheimer's disease, the Aβ(1-42) level is determined preferably in CSF or plasma. This level is decreased in AD patients, because of elevated aggregation of the AR peptides in the brain. It is surprising that the Aβ(1-40) concentration is—on the contrary—increased. Kim and co-workers have found a strong anti-amyloidogenic effect of Aβ(1-40) in vivo (Kim et al., 2007). They could show that increasing Aβ(1-40) levels in the brain of Tg2576 or BRI-Aβ42A mice protected against amyloid pathology. Moreover, the magnitude of this effect was quite unexpected: an approximately twofold increase of Aβ(1-40) levels in the forebrain of the BRI-Aβ40/Tg2576 mice had a lifelong inhibitory effect on Aβ deposition ranging from about 80% reduction at 11 months to about 50% at 20 months, compared with the Aβ deposition in Tg2576 littermates. Several other studies have supported this finding (Deng et al., 2006; Mucke et al., 2000, McGowan et el., 2005). It is imaginable that the increase of Aβ(1-40) level in plasma or CSF of AD patients is caused by a increased production in the brain as a consequence of elevated aggregation propensity of Aβ peptides to inhibit this unintended reaction.

The correlation of the plasma Aβ(1-40) concentration with the neuropsychological tests demonstrated herein, which became possible only by application of the new method of the present invention has shown that the significant increase of Aβ(1-40) level is an early event in the progression of Alzheimer's disease. Thereby, the level of plasma Aβ(1-40) can now be used as a marker for diagnosis of the onset of Alzheimer's disease.

The use of an internal plasma standard (ITS) analyzed together with unknown plasma samples within every measurement cycle as demonstrated herein further increases the reliability and comparability of the determined Aβ(1-40) levels, which were determined in different measurement cycles.

### REFERENCES

Blennow K, de Leon M J, Zetterberg H. Alzheimer's disease. Lancet. 2006 July 29;368(9533):387-403

Ferri C P, Prince M, Brayne C, Brodaty H, Fratiglioni L, Ganguli M, Hall K, Hasegawa K, Hendrie H, Huang Y, Jorm A, Mathers C, Menezes P R, Rimmer E, Scazufca M Global prevalence of dementia: a Delphi consensus study. Lancet. 2005 Dec. 17; 366(9503):2112-7

Knopman D S, DeKosky S T, Cummings J L, Chui H, Corey-Bloom J, Relkin N, Small G W, Miller B, Stevens J C. Practice parameter: diagnosis of dementia (an evidence-based review). Report of the Quality Standards Subcommittee of the American Academy of Neurology. Neurology. 2001 May 8; 56(9):1143-53

Waldemar G, Dubois B, Emre M, Georges J, McKeith I G, Rossor M, Scheltens P, Tariska P, Winblad B; EFNS. Recommendations for the diagnosis and management of Alzheimer's disease and other disorders associated with dementia: EFNS guideline. Eur J Neurol. 2007 January; 14(1):26.

Dubois B, Feldman H H, Jacova C, Dekosky S T, Barberger-Gateau P, Cummings J, Delacourte A, Galasko D, Gauthier S, Jicha G, Meguro K, O'brien J, Pasquier F, Robert P, Rossor M, Salloway S, Stern Y, Visser P J, Scheltens P. Research criteria for the diagnosis of Alzheimer's disease: revising the NINCDS-ADRDA criteria. Lancet Neurol. 2007 August; 6(8):734-46.

Jellinger K A. Alzheimer's disease. In: Gilman S, editor. Neurobiology of Disease. Amsterdam: Elsevier Academic Press; 2007. p. 69-82

Petersen R C, Smith G E, Waring S C, Ivnik R J, Tangalos E G, Kokmen E. Mild cognitive impairment: clinical characterization and outcome. Arch Neurol. 1999 March; 56(3):303-8

Chertkow H, Massoud F, Nasreddine Z, Belleville S, Joanette Y, Bocti C, Drolet V, Kirk J, Freedman M, Bergman H. Diagnosis and treatment of dementia: 3. Mild cognitive impairment and cognitive impairment without dementia. CMAJ. 2008 May 6; 178(10):1273-85

Scheff S W, Price D A, Schmitt F A, Mufson E J. Hippocampal synaptic loss in early Alzheimer's disease and mild cognitive impairment. Neurobiol Aging. 2006 October; 27(10):1372-84

Markesbery W R, Schmitt F A, Kryscio R J, Davis D G, Smith C D, Wekstein D R. Neuropathologic substrate of mild cognitive impairment. Arch Neurol. 2006 January; 63(1):38-46.

Bouwman F H, Schoonenboom S N, van der Flier W M, van Elk E J, Kok A, Barkhof F, Blankenstein M A, Scheltens P. CSF biomarkers and medial temporal lobe atrophy predict dementia in mild cognitive impairment. Neurobiol Aging. 2007 July; 28(7):1070-4

Saito Y, Murayama S. Neuropathology of mild cognitive impairment. Neuropathology 2007 December; 27(6):578-84

Jicha G A, Parisi J E, Dickson D W, Johnson K, Cha R, Ivnik R J, Tangalos E G, Boeve B F, Knopman D S, Braak H, Petersen R C. Neuropathologic outcome of mild cognitive impairment following progression to clinical dementia. Arch Neurol. 2006 May; 63(5):674-81

Petersen R C, Parisi J E, Dickson D W, Johnson K A, Knopman D S, Boeve B F, Jicha G A, Ivnik R J, Smith G E, Tangalos E G, Braak H, Kokmen E. Neuropathologic features of amnestic mild cognitive impairment. Arch Neurol. 2006 May; 63(5):665-72

Gauthier S, Reisberg B, Zaudig M, Petersen R C, Ritchie K, Broich K, Belleville S, Brodaty H, Bennett D, Chertkow H, Cummings J L, de Leon M, Feldman H, Ganguli M, Hampel H, Scheltens P, Tierney M C, Whitehouse P, Winblad B; International Psychogeriatric Association Expert Conference on mild cognitive impairment. Mild cognitive impairment. Lancet. 2006 Apr. 15; 367(9518):1262-70

Fischer P, Jungwirth S, Zehetmayer S, Weissgram S, Hoenigschnabl S, Gelpi E, Krampla W, Tragi K H. Conversion from subtypes of mild cognitive impairment to Alzheimer dementia. Neurology. 2007 Jan. 23; 68(4):288-91

Devanand D P, Pradhaban G, Liu X, Khandji A, De Santi S, Segal S, Rusinek H, Pelton G H, Honig L S, Mayeux R, Stern Y, Tabert M H, de Leon M J. Hippocampal and entorhinal atrophy in mild cognitive impairment: prediction of Alzheimer disease. Neurology. 2007 Mar. 13; 68(11): 828-36

Rossi R, Geroldi C, Bresciani L, Testa C, Binetti G, Zanetti O, Frisoni G B. Clinical and neuropsychological features associated with structural imaging patterns in patients with mild cognitive impairment. Dement Geriatr Cogn Disord. 2007; 23(3):175-83

Whitwell J L, Petersen R C, Negash S, Weigand S D, Kantarci K, Ivnik R J, Knopman D S, Boeve B F, Smith G E, Jack C R Jr. Patterns of atrophy differ among specific subtypes of mild cognitive impairment. Arch Neurol. 2007 August; 64(8):1130-8

Panza F, Capurso C, D'Introno A, Colacicco A M, Capurso A, Solfrizzi V. Heterogeneity of mild cognitive impairment and other predementia syndromes in progression to dementia. Neurobiol Aging. 2007 October; 28(10):1631-2; discussion 1633-4

Hyman S E. Can neuroscience be integrated into the DSM-V? Nat Rev Neurosci. 2007 September; 8(9):725-32

Blennow K. Cerebrospinal fluid protein biomarkers for Alzheimer's disease. NeuroRx. 2004 April; 1(2):213-25

Blennow K. CSF biomarkers for Alzheimer's disease: use in early diagnosis and evaluation of drug treatment. Expert Rev Mol Diagn. 2005 September; 5(5):661-72

Hampel H, Buerger K. Biomarkers in blood and cerebrospinal fluid. In: Herholz K, Morris C, Perani D, editors. The Dementias: Early Diagnosis and Evaluation. New York: Taylor & Francis; 2006. p. 73-107

Lewczuk P, Komhuber J, Wiltfang J. The German Competence Net Dementias: standard operating procedures for the neurochemical dementia diagnostics. J Neural Transm. 2006 August; 113(8):1075-80

Irizarry M C. Biomarkers of Alzheimer disease in plasma. NeuroRx. 2004 April; 1(2):226-34

Barnes J, Foster J, Fox N C. Structural magnetic resonace imigaing-derived biomarkers for Alzheimer's disaese. Biomarkers Med. 2007; 1: 79-92

Vemuri P, Gunter J L, Senjem M L, Whitwell J L, Kantarci K, Knopman D S, Boeve B F, Petersen R C, Jack C R Jr. Alzheimer's disease diagnosis in individual subjects using structural MR images: validation studies. Neuroimage. 2008 Feb. 1; 39(3):1186-97

Barkhof F, Polvikoski T M, van Straaten E C, Kalaria R N, Sulkava R, Aronen H J, Niinistö L, Rastas S, Oinas M, Scheltens P, Erkinjuntti T. The significance of medial temporal lobe atrophy: a postmortem MRI study in the very old. Neurology. 2007 Oct. 9; 69(15):1521-7

Mevel K, Desgranges B, Baron J C, Landeau B, De la Sayette V, Viader F, Eustache F, Chételat G. Detecting hippocampal hypometabolism in Mild Cognitive Impairment using automatic voxel-based approaches. Neuroimage. 2007 Aug. 1; 37(1):18-25

Kemppainen N M, Aalto S, Wilson I A, N\dot{a}gren K, Helin S, Brück A, Oikonen V, Kailajärvi M, Scheinin M, Viitanen M, Parkkola R, Rinne J O. PET amyloid ligand [11C]PIB uptake is increased in mild cognitive impairment. Neurology. 2007 May 8; 68(19):1603-6

Klunk W E, Engler H, Nordberg A, Wang Y, Blomqvist G, Holt D P, Bergstrom M, Savitcheva I, Huang G F, Estrada S, Ausén B, Debnath M L, Barletta J, Price J C, Sandell J,

Lopresti B J, Wall A, Koivisto P, Antoni G, Mathis C A, L\dot{a}ngström B. Imaging brain amyloid in Alzheimer's disease with Pittsburgh Compound-B. Ann Neurol. 2004 March; 55(3):306-19

Rowe C C, Ng S, Ackermann U, Gong S J, Pike K, Savage G, Cowie T F, Dickinson K L, Maruff P, Darby D, Smith C, Woodward M, Merory J, Tochon-Danguy H, O'Keefe G, Klunk W E, Mathis C A, Price J C, Masters C L, Villemagne V L. Imaging beta-amyloid burden in aging and dementia. Neurology. 2007 May 15; 68(20):1718-25

Pike K E, Savage G, Villemagne V L, Ng S, Moss S A, Maruff P, Mathis C A, Klunk W E, Masters C L, Rowe C C. Beta-amyloid imaging and memory in non-demented individuals: evidence for preclinical Alzheimer's disease. Brain. 2007 November; 130(Pt 11):2837-44

Huang C, Eidelberg D, Habeck C, Moeller J, Svensson L, Tarabula T, Julin P. Imaging markers of mild cognitive impairment: multivariate analysis of CBF SPECT. Neurobiol Aging. 2007 July; 28(7):1062-9

Kantarci K, Weigand S D, Petersen R C, Boeve B F, Knopman D S, Gunter J, Reyes D, Shiung M, O'Brien P C, Smith G E, Ivnik R J, Tangalos E G, Jack C R Jr. Longitudinal 1H MRS changes in mild cognitive impairment and Alzheimer's disease. Neurobiol Aging. 2007 September; 28(9):1330-9

Petrella J R, Wang L, Krishnan S, Slavin M J, Prince S E, Tran T T, Doraiswamy P M. Cortical deactivation in mild cognitive impairment: high-field-strength functional MR imaging. Radiology. 2007 October; 245(1):224-35

Hämäläinen A, Tervo S, Grau-Olivares M, Niskanen E, Pennanen C, Huuskonen J, Kivipelto M, Hänninen T, Tapiola M, Vanhanen M, Hallikainen M, Helkala E L, Nissinen A, Vanninen R, Soininen H. Voxel-based morphometry to detect brain atrophy in progressive mild cognitive impairment. Neuroimage. 2007 Oct. 1; 37(4):1122-31

Kircher T T, Weis S, Freymann K, Erb M, Jessen F, Grodd W, Heun R, Leube D T. Hippocampal activation in patients with mild cognitive impairment is necessary for successful memory encoding. J Neurol Neurosurg Psychiatry. 2007 August; 78(8):812-8

Kropholler M A, Boellaard R, van Berckel B N, Schuitemaker A, Kloet R W, Lubberink M J, Jonker C, Scheltens P, Lammertsma A A. Evaluation of reference regions for (R)-[(11)C]PK11195 studies in Alzheimer's disease and mild cognitive impairment. J Cereb Blood Flow Metab. 2007 December; 27(12):1965-74

Attems J. Sporadic cerebral amyloid angiopathy: pathology, clinical implications, and possible pathomechanisms. Acta Neuropathol. 2005 October; 110(4):345-59

Blennow K, Hampel H. CSF markers for incipient Alzheimer's disease. Lancet Neurol. 2003 October; 2(10):605-13

Hansson O, Zetterberg H, Buchhave P, Londos E, Blennow K, Minthon L. Association between CSF biomarkers and incipient Alzheimer's disease in patients with mild cognitive impairment: a follow-up study. Lancet Neurol. 2006 March; 5(3):228-34

Hansson O, Zetterberg H, Buchhave P, Andreasson U, Londos E, Minthon L, Blennow K. Prediction of Alzheimer's disease using the CSF Abeta42/Abeta40 ratio in patients with mild cognitive impairment. Dement Geriatr Cogn Disord. 2007;23(5):316-20

Fagan A M, Mintun M A, Mach R H, Lee S Y, Dence C S, Shah A R, LaRossa G N, Spinner M L, Klunk W E, Mathis C A, DeKosky S T, Morris J C, Holtzman D M. Inverse relation between in vivo amyloid imaging load and cerebrospinal fluid Abeta42 in humans. Ann Neurol. 2006 March; 59(3):512-9

Prince J A, Zetterberg H, Andreasen N, Marcusson J, Blennow K. APOE epsilon4 allele is associated with reduced cerebrospinal fluid levels of Abeta42. Neurology. 2004 Jun. 8; 62(11):2116-8

Strozyk D, Blennow K, White L R, Launer L J. CSF Abeta 42 levels correlate with amyloid-neuropathology in a population-based autopsy study. Neurology. 2003 Feb. 25; 60(4): 652-6

Walsh D M, Klyubin I, Shankar G M, Townsend M, Fadeeva J V, Betts V, Podlisny M B, Cleary J P, Ashe K H, Rowan M J, Selkoe D J. The role of cell-derived oligomers of Abeta in Alzheimer's disease and avenues for therapeutic intervention. Biochem Soc Trans. 2005 November; 33(Pt 5): 1087-90

Ewers M, Buerger K, Teipel S J, Scheltens P, Schroder J, Zinkowski R P, Bouwman F H, Schonknecht P, Schoonenboom N S, Andreasen N, Wallin A, DeBernardis J F, Kerkman D J, Heindl B, Blennow K, Hampel H. Multicenter assessment of CSF-phosphorylated tau for the prediction of conversion of MCI. Neurology. 2007 Dec. 11; 69(24):2205-12

Fagan A M, Roe C M, Xiong C, Mintun M A, Morris J C, Holtzman D M. Cerebrospinal fluid tau/beta-amyloid(42) ratio as a prediction of cognitive decline in nondemented older adults. Arch Neurol. 2007 March; 64(3):343-9.

Gustafson D R, Skoog I, Rosengren L, Zetterberg H, Blennow K. Cerebrospinal fluid beta-amyloid 1-42 concentration may predict cognitive decline in older women. J Neurol Neurosurg Psychiatry. 2007 May; 78(5):461-4

Li G, Sokal I, Quinn J F, Leverenz J B, Brodey M, Schellenberg G D, Kaye J A, Raskind M A, Zhang J, Peskind E R, Montine T J. CSF tau/Abeta42 ratio for increased risk of mild cognitive impairment: a follow-up study. Neurology. 2007 Aug. 14; 69(7):631-9

Stomrud E, Hansson O, Blennow K, Minthon L, Londos E. Cerebrospinal fluid biomarkers predict decline in subjective cognitive function over 3 years in healthy elderly. Dement Geriatr Cogn Disord. 2007; 24(2):118-24

Hampel H, Teipel S J, Fuchsberger T, Andreasen N, Wiltfang J, Otto M, Shen Y, Dodel R, Du Y, Farlow M, Möller H J, Blennow K, Buerger K. Value of CSF beta-amyloid1-42 and tau as predictors of Alzheimer's disease in patients with mild cognitive impairment. Mol Psychiatry. 2004 July; 9(7):705-10

Maccioni R B, Lavados M, Guillón M, Mujica C, Bosch R, Farias G, Fuentes P. Anomalously phosphorylated tau and Abeta fragments in the CSF correlates with cognitive impairment in MCI subjects. Neurobiol Aging. 2006 February; 27(2):237-44

Schönknecht P, Pantel J, Kaiser E, Thomann P, Schröder J. Increased tau protein differentiates mild cognitive impairment from geriatric depression and predicts conversion to dementia. Neurosci Lett. 2007 Apr. 6; 416(1):39-42

Clark C M, Xie S, Chittams J, Ewbank D, Peskind E, Galasko D, Morris J C, McKeel D W Jr, Farlow M, Weitlauf S L, Quinn J, Kaye J, Knopman D, Arai H, Doody R S, DeCarli C, Leight S, Lee V M, Trojanowski J Q. Cerebrospinal fluid tau and beta-amyloid: how well do these biomarkers reflect autopsy-confirmed dementia diagnoses? Arch Neurol. 2003 December; 60(12):1696-702

Buerger K, Ewers M, Pirttila T, Zinkowski R, Alafuzoff I, Teipel S J, DeBernardis J, Kerkman D, McCulloch C, Soininen H, Hampel H. CSF phosphorylated tau protein correlates with neocortical neurofibrillary pathology in Alzheimer's disease. Brain. 2006 November; 129(Pt 11):3035-41

Engelborghs S, Sleegers K, Cras P, Brouwers N, Serneels S, De Leenheir E, Martin J J, Vanmechelen E, Van Broeckhoven C, De Deyn P P. No association of CSF biomarkers with APOEepsilon4, plaque and tangle burden in definite Alzheimer's disease. Brain. 2007 September; 130(Pt 9):2320-6

Wiltfang J, Esselmann H, Bibl M, Hull M, Hampel H, Kessler H, Frölich L, Schröder J, Peters O, Jessen F, Luckhaus C, Perneczky R, Jahn H, Fiszer M, Maler J M, Zimmermann R, Bruckmoser R, Kornhuber J, Lewczuk P. Amyloid beta peptide ratio 42/40 but not A beta 42 correlates with phospho-Tau in patients with low- and high-CSF A beta 40 load. J Neurochem. 2007 May; 101(4):1053-9

Buerger K, Alafuzoff I, Ewers M, Pirttilä T, Zinkowski R, Hampel H. No correlation between CSF tau protein phosphorylated at threonine 181 with neocortical neurofibrillary pathology in Alzheimer's disease. Brain. 2007 October; 130(Pt 10):e82

Buerger K, Otto M, Teipel S J, Zinkowski R, Blennow K, DeBernardis J, Kerkman D, Schroder J, Schönknecht P, Cepek L, McCulloch C, Möller H J, Wiltfang J, Kretzschmar H, Hampel H. Dissociation between CSF total tau and tau protein phosphorylated at threonine 231 in Creutzfeldt-Jakob disease. Neurobiol Aging. 2006 January; 27(1):10-5

Bibl M, Mollenhauer B, Esselmann H, Schneider M, Lewczuk P, Welge V, Gross M, Falkai P, Kornhuber J, Wiltfang J. Cerebrospinal fluid neurochemical phenotypes in vascular dementias: original data and mini-review. Dement Geriatr Cogn Disord. 2008; 25(3):256-65

Zhong Z, Ewers M, Teipel S, Burger K, Wallin A, Blennow K, He P, McAllister C, Hampel H, Shen Y. Levels of beta-secretase (BACE1) in cerebrospinal fluid as a predictor of risk in mild cognitive impairment. Arch Gen Psychiatry. 2007 June; 64(6):718-26

McRae A, Martins R N, Fonte J, Kraftsik R, Hirt L, Miklossy J. Cerebrospinal fluid antimicroglial antibodies in Alzheimer disease: a putative marker of an ongoing inflammatory process. Exp Gerontol. 2007 April; 42(4):355-63

Jellinger K A, Janetzky B, Attems J, Kienzl E. Biomarkers for early diagnosis of Alzheimer disease: ‘ALZheimer ASsociated gene’—a new blood biomarker? J Cell Mol Med. 2008 August; 12(4):1094-117

Finehout E J, Franck Z, Choe L H, Relkin N, Lee K H. Cerebrospinal fluid proteomic biomarkers for Alzheimer's disease. Ann Neurol. 2007 February; 61(2):120-9

Castaño E M, Roher A E, Esh C L, Kokjohn T A, Beach T. Comparative proteomics of cerebrospinal fluid in neuropathologically-confirmed Alzheimer's disease and non-demented elderly subjects. Neurol Res. 2006 March; 28(2):155-63

Zhang J, Goodlett D R, Quinn J F, Peskind E, Kaye J A, Zhou Y, Pan C, Yi E, Eng J, Wang Q, Aebersold R H, Montine T J. Quantitative proteomics of cerebrospinal fluid from patients with Alzheimer disease. J Alzheimers Dis. 2005 April; 7(2):125-33

Simonsen A H, McGuire J, Hansson O, Zetterberg H, Podust V N, Davies H A, Waldemar G, Minthon L, Blennow K. Novel panel of cerebrospinal fluid biomarkers for the prediction of progression to Alzheimer dementia in patients with mild cognitive impairment. Arch Neurol. 2007 March; 64(3):366-70

Lescuyer P, Allard L, Zimmermann-Ivol C G, Burgess J A, Hughes-Frutiger S, Burkhard P R, Sanchez J C, Hochstrasser D F. Identification of post-mortem cerebrospinal fluid proteins as potential biomarkers of ischemia and neurodegeneration. Proteomics. 2004 August; 4(8):2234-41

Abdi F, Quinn J F, Jankovic J, McIntosh M, Leverenz J B, Peskind E, Nixon R, Nutt J, Chung K, Zabetian C, Samii A, Lin M, Hattan S, Pan C, Wang Y, Jin J, Zhu D, Li G J, Liu Y, Waichunas D, Montine T J, Zhang J. Detection of biomarkers with a multiplex quantitative proteomic platform in cerebrospinal fluid of patients with neurodegenerative disorders. J Alzheimers Dis. 2006 August; 9(3):293-348

Irani D N, Anderson C, Gundry R, Cotter R, Moore S, Kerr D A, McArthur J C, Sacktor N, Pardo C A, Jones M, Calabresi P A, Nath A. Cleavage of cystatin C in the cerebrospinal fluid of patients with multiple sclerosis. Ann Neurol. 2006 February; 59(2):237-47

Hansson S F, Simonsen A H, Zetterberg H, Andersen O, Haghighi S, Fagerberg I, Andréasson U, Westman-Brinkmalm A, Wallin A, Rüetschi U, Blennow K. Cystatin C in cerebrospinal fluid and multiple sclerosis. Ann Neurol. 2007 August; 62(2):193-6

Ravaglia G, Forti P, Maioli F, Chiappelli M, Montesi F, Tumini E, Mariani E, Licastro F, Patterson C. Blood inflammatory markers and risk of dementia: The Conselice Study of Brain Aging. Neurobiol Aging. 2007 December; 28(12):1810-20

Engelhart M J, Geerlings M I, Meijer J, Kiliaan A, Ruitenberg A, van Swieten JC, Stijnen T, Hofman A, Witteman J C, Breteler M M. Inflammatory proteins in plasma and the risk of dementia: the Rotterdam study. Arch Neurol. 2004 May; 61(5):668-72

Motta M, Imbesi R, Di Rosa M, Stivala F, Malaguarnera L. Altered plasma cytokine levels in Alzheimer's disease: correlation with the disease progression. Immunol Lett. 2007 Nov. 30; 114(1):46-51

German D C, Gurnani P, Nandi A, Garner H R, Fisher W, Diaz-Arrastia R, O'Suilleabhain P, Rosenblatt K P. Serum biomarkers for Alzheimer's disease: proteomic discovery. Biomed Pharmacother. 2007 August; 61(7):383-9

Ray S, Britschgi M, Herbert C, Takeda-Uchimura Y, Boxer A, Blennow K, Friedman L F, Galasko D R, Jutel M, Karydas A, Kaye J A, Leszek J, Miller B L, Minthon L, Quinn J F, Rabinovici G D, Robinson W H, Sabbagh M N, So Y T, Sparks D L, Tabaton M, Tinklenberg J, Yesavage J A, Tibshirani R, Wyss-Coray T. Classification and prediction of clinical Alzheimer's diagnosis based on plasma signaling proteins. Nat Med. 2007 November; 13(11): 1359-62

Graff-Radford N R, Crook J E, Lucas J, Boeve B F, Knopman D S, Ivnik R J, Smith G E, Younkin L H, Petersen R C, Younkin S G. Association of low plasma Abeta42/Abeta40 ratios with increased imminent risk for mild cognitive impairment and Alzheimer disease. Arch Neurol. 2007 March; 64(3):354-62

van Oijen M, Hofman A, Soares H D, Koudstaal P J, Breteler M M. Plasma Abeta(1-40) and Abeta(1-42) and the risk of dementia: a prospective case-cohort study. Lancet Neurol. 2006 August; 5(8): 655-60

Sundelof J, Giedraitis V, Irizarry M C, SundstrOm J, Ingelsson E, Rönnemaa E, Arnlov J, Gunnarsson M D, Hyman B T, Basun H, Ingelsson M, Lannfelt L, Kilander L. Plasma beta amyloid and the risk of Alzheimer disease and dementia in elderly men: a prospective, population-based cohort study. Arch Neurol. 2008 February; 65(2):256-63

Song M S, Mook-Jung I, Lee H J, Min J Y, Park M H. Serum anti-amyloid-beta antibodies and Alzheimer's disease in elderly Korean patients. J Int Med Res. 2007 May-June; 35(3):301-6

Pesaresi M, Lovati C, Bertora P, Mailland E, Galimberti D, Scarpini E, Quadri P, Forloni G, Mariani C. Plasma levels of beta-amyloid (1-42) in Alzheimer's disease and mild cognitive impairment. Neurobiol Aging. 2006 June; 27(6):904-5

Fukumoto H, Tennis M, Locascio J J, Hyman B T, Growdon J H, Irizarry M C. Age but not diagnosis is the main predictor of plasma amyloid beta-protein levels. Arch Neurol. 2003 July; 60(7):958-64

Kosaka T, Imagawa M, Seki K, Arai H, Sasaki H, Tsuji S, Asami-Odaka A, Fukushima T, Imai K, Iwatsubo T. The beta APP717 Alzheimer mutation increases the percentage of plasma amyloid-beta protein ending at A beta42(43). Neurology. 1997 March; 48(3):741-5

Scheuner D, Eckman C, Jensen M, Song X, Citron M, Suzuki N, Bird T D, Hardy J, Hutton M, Kukull W, Larson E, Levy-Lahad E, Viitanen M, Peskind E, Poorkaj P, Schellenberg G, Tanzi R, Wasco W, Lannfelt L, Selkoe D, Younkin S. Secreted amyloid beta-protein similar to that in the senile plaques of Alzheimer's disease is increased in vivo by the presenilin 1 and 2 and APP mutations linked to familial Alzheimer's disease. Nat Med. 1996 August; 2(8):864-70

Sobów T, Flirski M, Kloszewska I, Liberski P P. Plasma levels of alpha beta peptides are altered in amnestic mild cognitive impairment but not in sporadic Alzheimer's disease. Acta Neurobiol Exp (Wars). 2005; 65(2):117-24

Tamaoka A, Fukushima T, Sawamura N, Ishikawa K, Oguni E, Komatsuzaki Y, Shoji S. Amyloid beta protein in plasma from patients with sporadic Alzheimer's disease. J Neurol Sci. 1996 Sep. 15; 141(1-2):65-8

Vanderstichele H, Van Kerschaver E, Hesse C, Davidsson P, Buyse M A, Andreasen N, Minthon L, Wallin A, Blennow K, Vanmechelen E. Standardization of measurement of beta-amyloid(1-42) in cerebrospinal fluid and plasma. Amyloid. 2000 December; 7(4):245-58

Abdullah L, Paris D, Luis C, Quadros A, Parrish J, Valdes L, Keegan AP, Mathura V, Crawford F, Mullan M. The influence of diagnosis, intra- and inter-person variability on serum and plasma Abeta levels. Neurosci Lett. 2007 Nov. 27; 428(2-3):53-8

Freeman S H, Raju S, Hyman B T, Frosch M P, Irizarry M C. Plasma Abeta levels do not reflect brain Abeta levels. J Neuropathol Exp Neurol. 2007 April; 66(4):264-71

Mehta P D, Pirttila T, Patrick B A, Barshatzky M, Mehta S P. Amyloid beta protein 1-40 and 1-42 levels in matched cerebrospinal fluid and plasma from patients with Alzheimer disease. Neurosci Lett. 2001 May 18; 304(1-2):102-6

Giedraitis V, Sundelof J, Irizarry M C, GArevik N, Hyman B T, Wahlund L O, Ingelsson M, Lannfelt L. The normal equilibrium between CSF and plasma amyloid beta levels is disrupted in Alzheimer's disease. Neurosci Lett. 2007 Nov. 12; 427(3):127-31

Blasko I, Jellinger K, Kemmler G, Krampla W, Jungwirth S, Wichart I, Tragl K H, Fischer P. Conversion from cognitive health to mild cognitive impairment and Alzheimer's disease: prediction by plasma amyloid beta 42, medial temporal lobe atrophy and homocysteine. Neurobiol Aging. 2008 January; 29(1):1-11

Mehta PD, Pirttilä T, Mehta S P, Sersen E A, Aisen P S, Wisniewski H M. Plasma and cerebrospinal fluid levels of amyloid beta proteins 1-40 and 1-42 in Alzheimer disease. Arch Neurol. 2000 January; 57(1):100-5

Brettschneider S, Morgenthaler N G, Teipel S J, Fischer-Schulz C, Burger K, Dodel R, Du Y, Möller H J, Bergmann A, Hampel H. Decreased serum amyloid beta(1-42) autoantibody levels in Alzheimer's disease, determined by a newly developed immuneprecipitation assay with radiolabeled amyloid beta(1-42) peptide. Biol Psychiatry. 2005 Apr. 1; 57(7):813-6

Bibl M, Esselmann H, Mollenhauer B, Weniger G, Welge V, Liess M, Lewczuk P, Otto M, Schulz J B, Trenkwalder C, Kornhuber J, Wiltfang J. Blood-based neurochemical diagnosis of vascular dementia: a pilot study. J Neurochem. 2007 October; 103(2):467-74

Hansson O, Zetterberg H, Vanmechelen E, Vanderstichele H, Andreasson U, Londos E, Wallin A, Minthon L, Blennow K. Evaluation of plasma Abeta(40) and Abeta(42) as predictors of conversion to Alzheimer's disease in patients with mild cognitive impairment. Neurobiol Aging. 2008 May 16. [Epub ahead of print]

Kessler J, Calabrese P, Kalbe E, Berger F. Ein neues Screening-Verfahren zur Unterstützung der Demenzdiagnostik. Psycho 26 343-347 (2000)

Folstein M F, Folstein S E, McHugh P R. “Mini-mental state”. A practical method for grading the cognitive state of patients for the clinician. J Psychiatr Res. 1975 November; 12(3):189-98

Shulman K I, Shedletsky R, Silver I L. The challenge of time: Clock-drawing and cognitive function in the elderly. Int J Geriatr Psychiatry 1, 135-140 (1986)

lrie K, Murakami K, Masuda Y, Morimoto A, Ohigashi H, Ohashi R, Takegoshi K, Nagao M, Shimizu T, Shirasawa T. Structure of beta-amyloid fibrils and its relevance to their neurotoxicity: implications for the pathogenesis of Alzheimer's disease. J Biosci Bioeng. 2005 May; 99(5):437-47

Simonsen A H, Hansson S F, Ruetschi U, McGuire J, Podust V N, Davies H A, Mehta P, Waldemar G, Zetterberg H, Andreasen N, Wallin A, Blennow K. Amyloid beta1-40 quantification in CSF: comparison between chromatographic and immunochemical methods. Dement Geriatr Cogn Disord. 2007; 23(4):246-50

Casas C, Sergeant N, Itier J M, Blanchard V, Wirths O, van der Kolk N, Vingtdeux V, van de Steeg E, Ret G, Canton T, Drobecq H, Clark A, Bonici B, Delacourte A, Benavides J, Schmitz C, Tremp G, Bayer T A, Benoit P, Pradier L. Massive CA1/2 neuronal loss with intraneuronal and N-terminal truncated Abeta42 accumulation in a novel Alzheimer transgenic model. Am J Pathol. 2004 October; 165(4):1289-300

Gelfanova V, Higgs R E, Dean R A, Holtzman D M, Farlow M R, Siemers E R, Boodhoo A, Qian Y W, He X, Jin Z, Fisher D L, Cox K L, Hale J E. Quantitative analysis of amyloid-beta peptides in cerebrospinal fluid using immuneprecipitation and MALDI-Tof mass spectrometry. Brief Funct Genomic Proteomic. 2007 June; 6(2):149-58.

Sergeant N, Bombois S, Ghestem A, Drobecq H, Kostanjevecki V, Missiaen C, Wattez A, David J P, Vanmechelen E, Sergheraert C, Delacourte A. Truncated beta-amyloid peptide species in pre-clinical Alzheimer's disease as new targets for the vaccination approach. J Neurochem. 2003 June; 85(6):1581-91

Deng Y, Tarassishin L, Kallhoff V, Peethumnongsin E, Wu L, Li Y M, Zheng H. Deletion of presenilin 1 hydrophilic loop sequence leads to impaired gamma-secretase activity and exacerbated amyloid pathology. J Neurosci. 2006 Apr. 5; 26(14):3845-54.

Kim J, Onstead L, Randle S, Price R, Smithson L, Zwizinski C, Dickson D W, Golde T, McGowan E.

Abéta40 inhibits amyloid deposition in vivo. J Neurosci. 2007 Jan. 17; 27(3):627-33.

Mayeux R, Honig L S, Tang M X, Manly J, Stern Y, Schupf N, Mehta P D.

Plasma A[beta]40 and A[beta]42 and Alzheimer's disease: relation to age, mortality, and risk. Neurology. 2003 Nov. 11; 61(9):1185-90

McGowan E, Pickford F, Kim J, Onstead L, Eriksen J, Yu C, Skipper L, Murphy M P, Beard J, Das P, Jansen K, Delucia M, Lin W L, Dolios G, Wang R, Eckman C B, Dickson D W, Hutton M, Hardy J, Golde T.

Abeta42 is essential for parenchymal and vascular amyloid deposition in mice. Neuron. 2005 Jul. 21; 47(2):191-9.

Mehta P D, Pirttilä T, Mehta S P, Sersen E A, Aisen P S, Wisniewski H M.

Plasma and cerebrospinal fluid levels of amyloid beta proteins 1-40 and 1-42 in Alzheimer disease. Arch Neurol. 2000 Jan;57(1):100-5.

Mucke L, Masliah E, Yu G Q, Mallory M, Rockenstein E M, Tatsuno G, Hu K, Kholodenko D, Johnson-Wood K, McConlogue L.

High-level neuronal expression of abeta 1-42 in wild-type human amyloid protein precursor transgenic mice: synaptotoxicity without plaque formation.

J Neurosci. 2000 Jun. 1; 20(11):4050-8.

Simonsen A H, Hansson S F, Ruetschi U, McGuire J, Podust V N, Davies H A, Mehta P, Waldemar G, Zetterberg H, Andreasen N, Wallin A, Blennow K.

Amyloid beta1-40 quantification in CSF: comparison between chromatographic and immunochemical methods. Dement Geriatr Cogn Disord. 2007; 23(4):246-50.

van Oijen M, Hofman A, Soares H D, Koudstaal P J, Breteler M M.

Plasma Abeta(1-40) and Abeta(1-42) and the risk of dementia: a prospective case-cohort study. Lancet Neurol. 2006 August; 5(8):655-60.

What is claimed is:

1. A method for the detection of an Aβ target peptide in a biological sample, comprising the following steps:
i) contacting a biological sample with at least two different capture antibodies;
ii) detection of the resulting immune complex;
iii) destruction of the immune complex; and
iv) quantifying the captured Aβ peptides.

2. The method according to claim 1, wherein said method is performed in the presence of an internal standard sample.

3. The method according to claim 2, wherein said internal standard sample is selected from the group consisting of a plasma sample, cerebrospinal fluid sample , urine sample, lymph sample, saliva sample , sudor sample , pleura fluid sample, synovial fluid sample, aqueous fluid sample, tear fluid sample, bile sample, and pancreas secretion sample.

4. The method according to claim 3, wherein said internal standard sample is an internal standard plasma sample (ITS).

5. The method according to claim 2, wherein the method furthr comprises normalizing the Aβ target peptide concentrations, such as the Aβ(1-40) or the Aβ(1-42) concentrations, of a biological sample to the concentration of the respective Aβ target peptide determined in the internal standard sample, resulting in a relative Aβ target peptide level of each biological sample.

6. The method according to claim 1, wherein the Aβ target peptide is Aβ(1-40) including functional equivalents thereof

7. The method according to claim 1, wherein the Aβ target peptide is Aβ(1-40) of SEQ ID NO: 1 including functional equivalents thereof

8. The method according to claim 1, wherein the Aβ target peptide is Aβ(1-42) including functional equivalents thereof

9. The method according to claim 1, wherein the Aβ target peptide is Aβ(1-42) of SEQ ID NO: 2 including functional equivalents thereof

10. The method according to claim 1, wherein the biological sample is selected from the group consisting of blood, serum, urine, cerebrospinal fluid (CSF), plasma, lymph, saliva, sweat, pleural fluid, synovial fluid, tear fluid, bile and pancreas secretion.

11. The method according to claim 10, wherein the biological sample is plasma.

12. The method according to claim 1, wherein the at least two different capture antibodies are each specific for a different epitope on the Aβ target peptide.

13. The method according to claim 1, wherein the capture antibodies are selected from the group consisting of:
3D6, Epitope: 1-5,
pAb-EL16, Epitope: 1-7,
2H4, Epitope: 1-8,
1E11, Epitope: 1-8,
20.1, Epitope: 1-10,
Rabbit Anti-Aβ Polyclonal Antibody, Epitope: 1-14 (Abeam),
AB10, Epitope: 1-16,
82E1, Epitope: 1-16,
pAb1-42, Epitope: 1-11,
NAB228, Epitope: 1-11,
DE2, Epitope: 1-16,
DE2B4, Epitope: 1-17,
6E10, Epitope: 1-17,
10D5, Epitope: 3-7,
WO-2, Epitope: 4-10,
1A3, Epitope 5-9,
pAb-EL21, Epitope 5-11,
310-03, Epitope 5-16,
Chicken Anti-Human Aβ Polyclonal Antibody, Epitope 12-28 (Abeam),
Chicken Anti-Human Aβ Polyclonal Antibody, Epitope 25-35 (Abeam),
Rabbit Anti-Human Aβ Polyclonal Antibody, Epitope: N-terminal (ABR),
Rabbit Anti-Human Aβ Polyclonal Antibody (Anaspec),
12C3, Epitope 10-16,
16C9, Epitope 10-16,
19B8, Epitope 9-10,
pAb-EL26, Epitope: 11-26,
BAM90.1, Epitope: 13-28,
Rabbit Anti-beta-Amyloid (pan) Polyclonal Antibody, Epitope: 15-30 (MBL),
22D12, Epitope: 18-21,
266, Epitope: 16-24,
pAb-EL17; Epitope: 15-24,
4G8, Epitope: 17-24,
Rabbit Anti-Aβ Polyclonal Antibody, Epitope: 22-35 (Abeam)
G2-10; Epitope: 31-40,
Rabbit Anti-Aβ, aa 32-40 Polyclonal Antibody (GenScript Corporation),
EP1876Y, Epitope: x-40,
G2-11, Epitope: 33-42,
16C11, Epitope: 33-42,
21F12, Epitope: 34-42,
1A10, Epitope: 35-40, and
D-17 Goat anti-Aβ antibody, Epitope: C-terminal

14. The method according to claim 1, wherein the capture antibodies are selected from the group consisting of 3D6, BAN50, 82E1, 6E10, WO-2, 266, BAM90.1, 4G8, G2-10, 1A10, BA27, 11A5-B10, 12F4, and 21F12.

15. The method according to claim 1, wherein the following antibody pairs are used as capture antibodies:
4G8 and 11A5-B10,
3D6 and 4G8,
6E10 and 4G8,
82E1 and 4G8,
4G8 and 12F4,
4G8 and 21F12,
3D6 and 21F12,
6E10 and 21F12,
BAN50 and 4G8,
3D6 and 11A5-B10,
3D6 and 1A10,
3D6 and BA27,
6E10 and 11A5-B10,
6E10 and 1A10,
6E10 and BA27,
4G8 and 11A5-B10,
4G8 and 1A10,
4G8 and BA27,
4G8 and 12F4, and
4G8 and 21F12.

16. The method according to claim 1, wherein the detection of the complex is carried out by using secondary antibodies, specifically reacting with each capture antibody.

17. The method according to claim 16, wherein the secondary antibodies are anti-mouse antibodies or anti-rabbit antibodies.

18. The method according to claim 16, wherein the secondary antibodies are labelled.

19. The method according to claim 16, wherein the secondary antibodies are immobilized on magnetic beads.

20. The method according to claim 19, wherein the magnetic beads carrying the immune complex are separated from the biological sample using a magnetic separator.

21. The method according to claim 1, wherein the destruction of the immune complex is performed in the presence of 50% (v/v) Methanol/0.5% (v/v) formic acid.

22. The method according to claim 1, wherein the detected immune complex is quantified.

23. The method according to claim 22, wherein the captured Aβ peptides are quantified by a quantification means selected from the group consisting of sandwich ELISA, Amyloid β1-40 HTRF® Assay, Alphascreen™ Assay, Multiplex Assay Systems, mass spectrometry and Western Blot analysis.

24. The method according to claim 22, wherein the captured Aβ peptides are quantified by a sandwich ELISA as quantification means.

25. The method according to claim 24, wherein the sandwich ELISA comprises a first antibody, which is specific for the complete N-terminus of Aβ(1-40); and a detection antibody, which is specific for the C-terminus ending at amino acid 40 of Aβ(1-40).

26. The method according to claim 24, wherein the sandwich ELISA comprises a first antibody, which is specific for the complete N-terminus of Aβ(1-42); and a detection antibody, which is specific for the C-terminus ending at amino acid 42 of Aβ(1-42).

27. The method according to claim 24, wherein the sandwich ELISA comprises a first antibody, which is specific for the C-terminus of Aβ(1-40); and a detection antibody, which is specific for the complete N-terminus starting with Asp-Ala-Glu of Aβ(1-40).

28. The method according to claim 24, wherein the sandwich ELISA comprises a first antibody, which is specific for the C-terminus of Aβ(1-42); and a detection antibody, which is specific for the complete N-terminus starting with Asp-Ala-Glu of Aβ(1-42).

29. The method according to claim 25, wherein the first antibody is immobilized.

30. The method according to claim 25, wherein the detection antibody is labeled.

31. The method according to claim 24, wherein an ELISA-Kit for the quantification of Aβ(1-40) is used.

32. The method according to claim 31, wherein the ELISA-Kit is selected from the group consisting of Amyloid-β(1-40) (N) ELISA (IBL, JP27714); Aβ[1-40] Human ELISA Kit (Invitrogen); Human Amyloid beta (Amyloid-b), aa 1-40 ELISA Kit (Wako Chemicals USA, Inc.); and Amyloid Beta 1-40 ELISA Kit (The Genetics Company).

33. The method according to claim 24, wherein an ELISA-Kit for the quantification of Aβ(1-42) is used.

34. The method according to claim 33, wherein the ELISA-Kit is selected from the group consisting Amyloid-β(1-42) (N) ELISA (IBL, JP27712); Aβ[1-42] Human ELISA Kit (Invitrogen), Human Amyloid beta (Amyloid-β), aa 1-42 ELISA Kit (Wako Chemicals USA, Inc.), Amyloid Beta 1-40 ELISA Kit (The Genetics Company), INNOTEST® β-AMYLOID(1-42) (Innogenetics).

35. The method according to claim 1, wherein the state of the neurodegenerative disease of the subjects that are donors of the biological samples is characterized in one or more psychometric tests.

36. The method according to claim 35, wherein said psychometric tests are selected from the group consisting of DemTect Test, Mini-Mental-State Test, Clock-Drawing Test, ADAS-Cog, Blessed Test, CANTAB, Cognistat, NPI, BEHAVE-AD, CERAD, CSDD, GDS and the The 7 Minute Screen.

37. A method for diagnosing Alzheimer's disease comprising the method for the detection of an Aβ target peptide according to claim 1.

38. The method according to claim 37 for the differential diagnosis of Alzheimer's disease.

39. The method according to claim 37 for the diagnosis of early stages of Alzheimer' disease.

40. The method according to claim 39 for the diagnosis of Mild Cognitive Impairment.

41. An in vitro method for the diagnosis of Alzheimer's disease comprising the method for the detection of an Aβ target peptide according to claim 1.

42. A method for diagnosing Alzheimer's disease comprising an Aβ target peptide.

43. The method according to claim 42 for the differential diagnosis of Alzheimer's disease.

44. The method according to claim 41 for the diagnosis of early stages of Alzheimer' disease.

45. The method according to claim 44 for the diagnosis of Mild Cognitive Impairment.

46. The method according to claim 37, wherein the Aβ target peptide is Aβ(1-40) including functional equivalents thereof.

47. The method according to claim 37, wherein the Aβ target peptide is Aβ(1-42) including functional equivalents thereof.


SEQUENCE LISTING (2 sequences)

SEQ ID NO: 1 - protein, 42 aa, Homo sapiens [sequence omitted]

SEQ ID NO: 2 - protein, 40 aa, Homo sapiens
DAEFRHDSGYEVHHQKLVFFAEDVGSNKGAIIGLMVGGVV